(19) United States
(12) Patent Application Publication
(10) Pub. No.: US 2022/0226334 A1
(43) Pub. Date: Jul. 21, 2022

(54) ANTIBODY CONJUGATE AND APPLICATION OF PHARMACEUTICAL COMPOSITION THEREOF

(71) Applicants: SHANGHAI FUDAN-ZHANGJIANG BIO-PHARMACEUTICAL CO., LTD., Shanghai (CN); SHANGHAI JIAOLIAN MEDICINE RESEARCH AND DEVELOPMENT CO., LTD., Shanghai (CN)

(72) Inventors: Yijun SHEN, Shanghai (CN); Tong YANG, Shanghai (CN); Yanjun LIU, Shanghai (CN); Qingsong GUO, Shanghai (CN); Xuemei CAO, Shanghai (CN); Hua LI, Shanghai (CN); Bei GAO, Shanghai (CN); Guanghao WU, Shanghai (CN); Likai MENG, Shanghai (CN); Yanbo GAO, Shanghai (CN); Yang ZHU, Shanghai (CN)

(21) Appl. No.: 17/612,849

(22) PCT Filed: May 21, 2019
(86) PCT No.: PCT/CN2019/087831

§ 371 Date: Nov. 19, 2021

(51) Int. Cl. A61K 31/537; A61K 47/54; A61K 47/68

(52) U.S. Cl. CPC A61K 31/537; A61K 47/68; A61K 47/54

(57) ABSTRACT

An application of an antibody conjugate in the preparation of a drug for treating CD30 positive tumors, which is characterized in that: the antibody conjugate is F0002-ADC; the general formula of the structural thereof is Ab-Lₘ-Yₙ; and the CD30-positive tumors are CD30-positive tumors that express a multidrug resistance gene 1. In addition, a pharmaceutical combination and a pharmaceutical composition containing the antibody conjugate, which may be applied to the preparation of a drug used for treating CD30-positive tumors.

### TECHNICAL FIELD

The present disclosure relates to a use of an antibody conjugate and a pharmaceutical composition thereof.

### BACKGROUND

Lymphoma is the top ten malignant tumors in China in terms of morbidity and mortality (Cancer statistics in China, 2015. CA Cancer J Clin. 2016; 66(2):115-32.), according to the calculation of the National Cancer Center in 2015, there were 88,200 new cases of lymphoma and 52,100 deaths. Lymphoma is classified as Hodgkin lymphoma (HL) and non-Hodgkin lymphoma (NHL), 95% of HL is classical Hodgkin lymphoma (cHL), the incidence of HL in Europe and the United States is 2-3 cases per 100,000 people, accounting for about 20-30% of all lymphomas, which belongs to rare disease (Epidemiology and etiology of Hodgkin's lymphoma Ann Oncol. 2002; 13 Suppl 4:147-52), and HL in China is even less, accounting for only 8-9% of lymphomas, with an annual incidence of about 0.6 cases per 100,000 people. CD30 is the hallmark antigen of classical Hodgkin lymphoma. Anaplastic large cell lymphoma (ALCL), also known as ki-1 lymphoma, belongs to non-Hodgkin lymphoma with CD30-positive cells, accounting for 3-5% of all NHL and 10-20% of childhood lymphomas (J Clin Oncol 2008; 26(25):4124-30); ALCL is a highly malignant lymphoma without standardized chemotherapy scheme, and CHOP (Cyclophosphamide, Doxorubicin, Vincristine, and Prednisone) is most commonly used, with an ORR of about 70-80% and a 5-year survival rate of about 52%. The treatment can be carried out by radiotherapy, chemotherapy, bone marrow transplantation, etc. Chemotherapy is the most appropriate, most cases can be completely remitted (CR), the recurrence rate is low, and the 3-year and 5-year survival rates are both high. The initial effect of radiotherapy is good, but it is easy to relapse in the long term. Relapsed and refractory ALCL lack effective treatments. Cutaneous T-cell lymphoma (CTCL) mainly includes mycosis fungoides (MF) and Sezary syndrome (SS), etc. The incidence of CTCL is about 0.64 per 100,000 people, wherein the incidence of MF is about 0.4 per 100,000 people (Am J Hematol. 2016 January; 91(1):151-65). Nearly half of CTCL are CD30-positive, and most of these diseases have low malignancy and slow disease progress. However, due to the abnormal systemic immune system in the late stage, the probability of secondary infection and suffering from the second tumor increased significantly. At present, the disease cannot be cured, and the main goal of treatment is to maintain long-term remission. Although the first-line chemotherapy of HL, ALCL and CTCL is effective, there is no effective treatment for refractory and recurrent diseases, the launch of ADCETRIS, a CD30 target ADC drug, is a breakthrough in the treatment of these diseases in recent years.

According to the 2015 edition of the Chinese malignant lymphoma treatment guidelines, the first-line treatment scheme for classical Hodgkin lymphoma (cHL) is ABVD (Doxorubicin (Adriamycin), Bleomycin, Vincristine, and Dacarbazine) with good efficacy, yet long-term disease control is not achieved in 15-30% of cHL patients (N Engl J Med 2003; 348:2386-95. [Erratum, N Engl J Med 2005; 353:744), and although autologous stem cell transplantation therapy (ASCT) is available, ASCT is effective in only 50% of patients. 40-65% of ALCL recurred after first-line treatment, and nearly half of the patients were ineffective after second-line radiotherapy and chemotherapy; ADCETRIS, a CD30-targeted ADC drug, was the first ALCL-targeted drug.

As a member of tumor necrosis factor receptor superfamily, CD30 is the hallmark antigen of classical Hodgkin lymphoma (cHL) and anaplastic large cell lymphoma (ALCL). Based on the high expression of CD30 on HL and ALCL cells, Seattle Genetics, Inc. developed the CD30 antibody drug conjugate Brentuximab vedotin (brentuximab-VC-MMAE, trade name ADCETRIS, code name SGN-35) for clinical use in the second-line treatment of HL and ALCL, with a clinical effectiveness rate (ORR) reached 73% and 86%, respectively, and the treatment remission reached a mean of 6.7 and 12.6 months. In the monotherapy trial for relapsed or refractory classical Hodgkin lymphoma, 34 of 102 treated patients (33%) achieved complete remission, with 5-year overall survival valued at 41% and 5-year progression-free survival valued at 22%; patients who achieved complete remission had 5-year survival valued at 64% and 5-year progression-free survival valued at 52% (also effective to the relapse in those previously effective), the relapsed or refractory CD30-positive HL and ALCL are effectively inhibited, and it was approved for marketing by the FDA in August 2011, being a targeted new drug that was the first to be approved by the FDA for the treatment of Hodgkin lymphoma since 1977 and the first to be specifically used for the treatment of ALCL (N Engl J Med 2010; 363:1812-21).

In August 2015, the FDA approved ADCETRIS for an expanded indication for patients with Hodgkin lymphoma (HL) at high risk of relapse after receiving stem cell transplantation. ADCETRIS has become the only consolidated treatment plan approved by FDA at present, which would help HL patients maintain remission after stem cell transplantation.

On Nov. 10, 2017, the FDA approved ADCETRIS for the treatment of patients with cutaneous T-cell lymphoma (CTCL) who have received prior systemic therapy, specifically for the treatment of adult patients who develop primary cutaneous anaplastic large cell lymphoma (pcALCL) and mycosis fungoides (MF) expressing CD30. Research data showed that compared with the standard treatment group (Methotrexate or Bexarotene), the Brentuximab vedotin group showed a statistically significant improvement in objective response rates lasting at least 4 months (56.3% vs. 12.5%) and a significant improvement in complete remission rates and progression-free survival (17 months vs. 4 months) (Lancet. 2017; 390(10094):555-66).

In recent years, some progress has been made in the treatment of HL with PD1 immunocheckpoint inhibitor, but its clinical efficacy is not as good as that of ADCETRIS, and it is only used for salvage treatment after the progress of existing treatment or the ineffectiveness of various treatment schemes. ADCETRIS, a CD30 target antibody drug conjugate, has become the most effective therapeutic drug for refractory and recurrent HL, ALCL, CTCL and other fields. ADCETRIS has achieved great success in clinical practice, but its toxic and side effects are found to be strong in clinical use, and the patient's tolerance is poor; according to the FDA application data Clinical Pharmacy Review, in phase II clinical practice of ADCETRIS, the incidence of AE events above Grade 3 was 55%, SAE events were 30%, and intolerance withdrawal was 20% (the application data of Adcetris EMA), and many treatment-related deaths occurred during the clinical period. Clinical use is limited to a maximum of 16 cycles; in January 2012, FDA released drug safety information, informing the public of 2 cases of multifocal leukoencephalopathy (PML) related to the lymphoma treatment drug brentuximab vedotin (ADCETRIS), which is a rare and serious brain infection and can lead to death. Due to the serious nature of PML, a black box warning has been added to the drug label. Serious side effects and intolerance to ADCETRIS limit the use of ADCETRIS. The reason for the strong toxic and side effects of ADCETRIS is the “bystander effect” caused by MMAE. The ADCETRIS structure comprises three components: an antibody cAC10 targeting CD30, an enzymatically degradable valine citrulline dipeptide (VC) linker, and a highly active microtubule protein inhibitor MMAE.

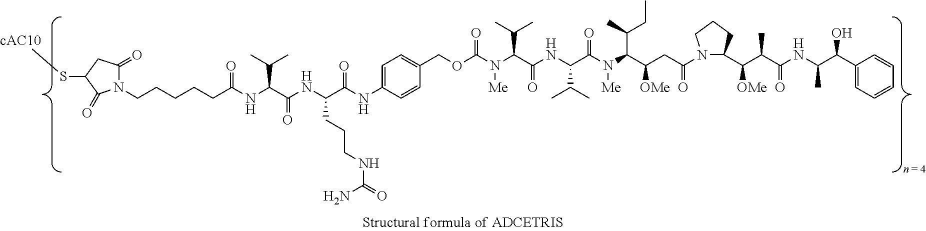

ADCETRIS binds to the cell membrane surface CD30 to endocytose into the cell, and enzymatically cleaves the VC linker specifically through histone B in the lysosome, thus releasing the active molecule MMAE, which prevents the polymerization of microtubulin, inhibits cell mitosis, and kills tumor cells. However, MMAE has good cell permeability and can enter other cells in the region after being released from apoptotic cells, thus creating a “bystander effect”, i.e., killing CD30-specific tumor cells and then killing surrounding cells non-specifically, resulting in significant toxic side effects in clinical practice.

At present, the first-line treatment of advanced-stage cHL mainly includes ABVD and BEACOPP schemes, with complete remission rates of 72% and 90% respectively (New England Journal of Medicine, 2018, 378 (4): 331-344.), although BEACOPP scheme has a high complete remission rate, its clinical application has high toxic and side effects, which largely limits the application of BEACOP scheme. The ABVD scheme is the mainstream chemotherapy scheme in different pathological stages of cHL at present, but Bleomycin is highly toxic and has unpredictable pulmonary toxicity BPT (Bleomycin pulmonary toxicity), which threatens life safety, thus affecting the overall therapeutic index of ABVD scheme; a retrospective study found that the removal of Bleomycin in ABVD scheme at any time point in clinical practice had no significant effect on the effectiveness, safety and recurrence rate of the overall scheme (J Clin Oncol, 2004, 22 (8): 1532-1533).

The phase II clinical results of Adcetris in second-line treatment of relapsed and refractory cHL patients show that it is very effective and its toxic and side effects are clearly controllable (Journal of Clinical Oncology, 2012, 30 (18): 2183-2189). Preclinical studies have shown synergistic effects of Adcetris in combination with ABVD schemes for cHL models (British Journal of Haematology, 2008, 142(1): 69-73), so the feasibility of Adcetris in combination with ABVD schemes for first-line treatment was investigated in early clinical trials; the results showed that Adcetris in combination with ABVD had no significant effect on pulmonary infections, but its pulmonary toxicity was significantly higher compared to ABVD (44% vs. 25%), reducing the therapeutic index of the combination. In view of the potential pulmonary toxicity of Bleomycin and the fact that the removal of Bleomycin has no effect on ABVD scheme as a whole, it is expected that a new first-line chemotherapy scheme, A+AVD, formed by replacing Bleomycin with Adcetris will gain better clinical benefits, thus improving therapeutic index.

### Content of the Present Invention

The purpose of the present disclosure is to solve the problems of drug resistance, high cytotoxicity and inability to combine in the prior art for the treatment of CD30-positive tumors, and to provide a use of an antibody conjugate and a pharmaceutical composition thereof. The antibody conjugate and the pharmaceutical composition thereof can be used for the preparation of a medicament for treating CD30-positive tumors expressing multidrug resistance gene 1.

To achieve the above object of the present disclosure, one of the technical solutions of the present disclosure is: the present disclosure provides a use of an antibody conjugate in the preparation of a medicament for the treatment of CD30-positive tumors; the antibody conjugate is F0002-ADC with a structural general formula of Ab-Lₘ-Yₙ: the CD30-positive tumor is CD30-positive tumor expressing multidrug resistance gene 1;

wherein, Ab is an anti-human CD30 antibody cAC10, an active fragment thereof, or a variant thereof;

the Ab is only connected with the L;

Y is Mertansine as shown in formula DM1;

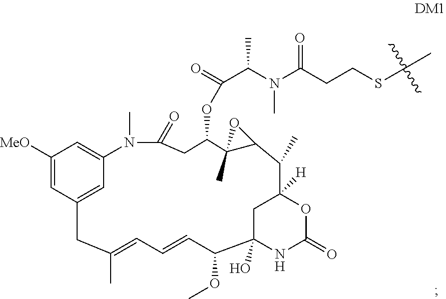

the Y is only connected with the L;

m is 3.3-10; n is 3.3-3.9; and m≥n; (when m>n, it means that both ends of part of the L are respectively connected with the Ab and the Y, and the rest of L is only connected with the Ab;)

when both ends of the L are respectively connected with the Ab and the Y, the L is

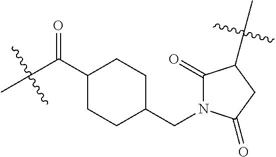 [SMILES: *C(=O)C1CCC(CN2C(=O)CC(*)C2=O)CC1]

(i.e., MCC linker), its left end forms an amide bond with the amino in lysine of the Ab, and its right end forms a thioether bond with S in the DM1;

when the L is only connected with the Ab, the L is

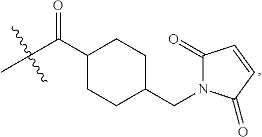 [SMILES: *C(=O)C1CCC(CN2C(=O)C=CC2=O)CC1]

and its left end forms an amide bond with the amino in lysine of the Ab.

Preferably, the m is equal to the n, the general structural formula is Ab-(L-Y)n, and

the structure is as follows:

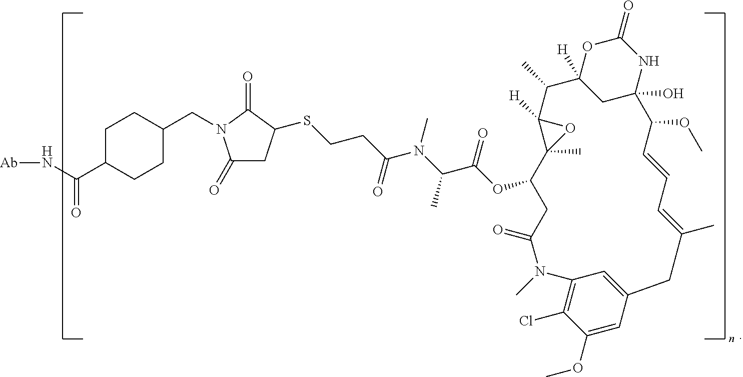

More preferably, the similarity between the variant of the anti-human CD30 antibody cAC10 and the amino acid sequence of cAC10 is not less than 90% (e.g., 90%, 92%, 93%, 94%, 95%, 96% or 97%), and the mutations related to lysine is not more than 80%.

Further preferably, n=3.6;

more preferably, the distribution of different DAR values is as follows:

| D0 | D1 | D2 | D3 | D4 | D5 | D6 | D7 |
|---|---|---|---|---|---|---|---|
| 3% | 10% | 17% | 20% | 18% | 16% | 9% | 7% |

In a preferred embodiment of the present disclosure, in the F0002-ADC, the m is equal to the n, the general structural formula is Ab-(L-Y)n, and the structure is as follows: is the following structure:

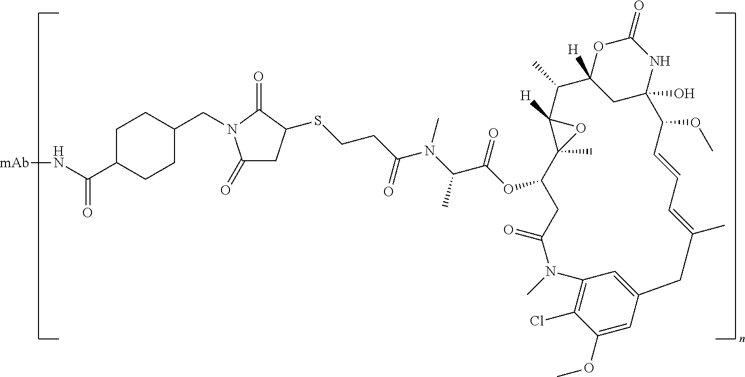

The distribution of different DAR values is as follows:

| D0 | D1 | D2 | D3 | D4 | D5 | D6 | D7 |
|---|---|---|---|---|---|---|---|
| 3% | 10% | 17% | 20% | 18% | 16% | 9% | 7% |

n=3.6.

In a preferred embodiment of the present disclosure, the CD30-positive tumor expressing multidrug resistance gene 1 (MDR1) may be a CD30-positive Hodgkin lymphoma expressing multidrug resistance gene 1 (its cell is, e.g., CD30-positive Hodgkin lymphoma cells L428 expressing multidrug resistance gene 1 or CD30-positive Hodgkin lymphoma cells L540 expressing multidrug resistance gene 1).

The present disclosure also provides a use of an antibody conjugate in the preparation of a medicament for the treatment of CD30-positive tumors; the antibody conjugate is the F0002-ADC; the CD30-positive tumor is CD30-positive tumor resistant to Adcetris. Preferably, the CD30-positive tumor resistant to Adcetris is CD30-positive Hodgkin lymphoma resistant to Adcetris (its cell is, e.g., CD30-positive Hodgkin lymphoma cells L428 resistant to Adcetris or CD30-positive Hodgkin lymphoma cells L540 resistant to Adcetris).

The present disclosure also provides a use of an antibody conjugate in the preparation of a medicament for the treatment of CD30-positive tumors; the antibody conjugate is the F0002-ADC; the CD30-positive tumor is CD30-positive Hodgkin lymphoma. Preferably, the CD30-positive Hodgkin lymphoma cell is CD30-positive Hodgkin lymphoma cell L428 or CD30-positive Hodgkin lymphoma cell L540.

The present disclosure provides a method for the treatment of CD30-positive tumors by administering an effective dose of the F0002-ADC to a patient; the CD30-positive tumor is CD30-positive tumor expressing multidrug resistance gene 1, or, CD30-positive tumor resistant to Adcetris, or, CD30-positive Hodgkin lymphoma.

In some embodiments, the CD30-positive lymphoma is CD30-positive Hodgkin lymphoma, CD30-positive anaplastic large cell lymphoma, CD30-positive diffuse histiocytic lymphoma or CD30-positive cutaneous T cell lymphoma; the most preferably, the CD30-positive lymphoma is CD30-positive Hodgkin lymphoma (its cell is, e.g., CD30-positive Hodgkin lymphoma cells L428 or CD30-positive Hodgkin lymphoma cells L540).

In some embodiments, the CD30-positive tumor expressing multidrug resistance gene 1 is CD30-positive Hodgkin lymphoma expressing multidrug resistance gene 1 (its cell is, e.g., CD30-positive Hodgkin lymphoma cells L428 expressing multidrug resistance gene 1 or CD30-positive Hodgkin lymphoma cells L540 expressing multidrug resistance gene 1).

In some embodiments, the CD30-positive tumor resistant to Adcetris is CD30-positive Hodgkin lymphoma resistant to Adcetris (its cell is, e.g., CD30-positive Hodgkin lymphoma cells L428 resistant to Adcetris or CD30-positive Hodgkin lymphoma cells L540 resistant to Adcetris).

On the other hand, the present disclosure provides a pharmaceutical combination comprising an antibody conjugate X and substance Y;

the antibody coupling X is the F0002-ADC; the substance Y is one or more of substances Y1, Y2, Y3, Y4, Y5, Y6, Y7 and Y8;

The substance Y1 is Y1-1, Y1-2 or Y1-3; Y1-1 is Doxorubicin, a pharmaceutically acceptable salt thereof, a solvate thereof, or, a solvate of the pharmaceutically acceptable salt thereof (e.g. Doxorubicin hydrochloride); Y1-2 is Epirubicin, a pharmaceutically acceptable salt thereof, a solvate thereof, or, a solvate of the pharmaceutically acceptable salt thereof, Y1-3 is Daunorubicin, a pharmaceutically acceptable salt thereof, a solvate thereof, or, a solvate of the pharmaceutically acceptable salt thereof.

The substance Y2 is Y2-1, Y2-2, Y2-3, Y2-4 or Y2-5; Y2-1 is Bleomycin, a pharmaceutically acceptable salt thereof, a solvate thereof, or, a solvate of the pharmaceutically acceptable salt thereof; Y2-2 is Boanmycin, a pharmaceutically acceptable salt thereof, a solvate thereof, or, a solvate of the pharmaceutically acceptable salt thereof; Y2-3 is Boningmycin, a pharmaceutically acceptable salt thereof, a solvate thereof, or, a solvate of the pharmaceutically acceptable salt thereof, Y2-4 is Pingyangmycin, a pharmaceutically acceptable salt thereof, a solvate thereof, or, a solvate of the pharmaceutically acceptable salt thereof, Y2-5 is Peplomycin, a pharmaceutically acceptable salt thereof, a solvate thereof, or, a solvate of the pharmaceutically acceptable salt thereof.

The substance Y3 is Y3-1, Y3-2, Y3-3 or Y3-4; Y3-1 is Vinblastine, a pharmaceutically acceptable salt thereof, a solvate thereof, or, a solvate of the pharmaceutically acceptable salt thereof, Y3-2 is Vincristine, a pharmaceutically acceptable salt thereof, a solvate thereof, or, a solvate of the pharmaceutically acceptable salt thereof; Y3-3 is Vinorelbine, a pharmaceutically acceptable salt thereof, a solvate thereof, or, a solvate of the pharmaceutically acceptable salt thereof; Y3-4 is Vindesine, a pharmaceutically acceptable salt thereof, a solvate thereof, or, a solvate of the pharmaceutically acceptable salt thereof.

The substance Y4 is Y4-1 or Y4-2; Y4-1 is Dacarbazine, a pharmaceutically acceptable salt thereof, a solvate thereof, or, a solvate of the pharmaceutically acceptable salt thereof, Y4-2 is Temozolomide, a pharmaceutically acceptable salt thereof, a solvate thereof, or, a solvate of the pharmaceutically acceptable salt thereof.

The substance Y5 is Y5-1 or Y5-2; Y5-1 is Etoposide, a pharmaceutically acceptable salt thereof, a solvate thereof, or, a solvate of the pharmaceutically acceptable salt thereof, Y5-2 is Teniposide, a pharmaceutically acceptable salt thereof, a solvate thereof, or, a solvate of the pharmaceutically acceptable salt thereof;

The substance Y6 is Y6-1 or Y6-2; Y6-1 is Cyclophosphamide, a pharmaceutically acceptable salt thereof, a solvate thereof, or, a solvate of the pharmaceutically acceptable salt thereof, Y6-2 is Ifosfamide, a pharmaceutically acceptable salt thereof, a solvate thereof, or, a solvate of the pharmaceutically acceptable salt thereof.

The substance Y7 is Procarbazine, a pharmaceutically acceptable salt thereof, a solvate thereof, or, a solvate of the pharmaceutically acceptable salt thereof.

The substance Y8 is Y8-1 or Y8-2; Y8-1 is Prednisone, a pharmaceutically acceptable salt thereof, a solvate thereof, or, a solvate of the pharmaceutically acceptable salt thereof, Y8-2 is Prednisone, a pharmaceutically acceptable salt thereof, a solvate thereof, or, a solvate of the pharmaceutically acceptable salt thereof.

In some embodiments, the substances Y are substances Y1, Y3 and Y4; preferably Y1-1, Y3-2 and Y4-1 (i.e. F0002-ADC+AVD scheme); more preferably the molar ratio of the antibody conjugates X:Y1-1:Y3-2:Y4-1 is 1:(400-800):(11-400):(550000-3000000) (e.g., 1:400:400:3000000, 1:800:30000:11:550000).

In some embodiments, the substances Y are substances Y1, Y2, Y3 and Y4; preferably Y1-1, Y2-1, Y3-2 and Y4-1 (i.e. F0002-ADC+ABVD scheme); more preferably the molar ratio of the antibody conjugates X:Y1-1:Y2-1:Y3-2:Y4-1 is 1:(400-800):(30000-45000):(11-400):(550000-3000000) (e.g., 1:800:45000:11:550000, 1:400:30000:400:3000000).

In some embodiments, the substances Y are substances Y2, Y5, Y1, Y6, Y3, Y7 and Y8; preferably Y2-1, Y5-1, Y1-1, Y6-1, Y3-2, Y7-1 and Y8-1 (i.e. F0002-ADC+BEACOPP scheme); more preferably the molar ratio of the antibody conjugates X:Y2-1:Y5-1:Y1-1:Y6-1:Y3-2:Y7-1:Y8-1 is 1:30000:700000:400:8000000:400:6500000:1500000.

In some embodiments, the substances Y are substances Y6, Y1, Y3 and Y8; preferably Y6-1, Y1-1, Y3-2 and Y8-1 (i.e. F0002-ADC+CHOP scheme); more preferably the molar ratio of the antibody conjugates X:Y6-1:Y1-1:Y3-2:Y8-1 is 1:(1700000-12000000):(800-1200):(11-600):(550000-1400000) (e.g., 1:1700000:800:11:1400000, 1:12000000:1200:600:5500000).

In some embodiments, the substances Y are substances Y6, Y3 and Y8; preferably Y6-1, Y3-1 and Y8-1 (i.e. F0002-ADC+CVP scheme); more preferably the molar ratio of the antibody conjugates X:Y6-1:Y3-1:Y8-1 is 1:(8000000-12000000):(400-600):(1500000-5500000) (e.g., 1:8000000:400:1500000,1:12000000:600:5500000).

In some embodiments, according to needs, the pharmaceutical combination of the present disclosure can be in the form of a mixture of all components, or in the form that each component is independent, or in the form that each component is divided into several groups (mixed in groups).

In the pharmaceutical combination, the antibody conjugate X and “all or part of the substance Y” may be administered simultaneously or separately.

In the “all or part of the substance Y”, e.g., when substance Y is Y1 and Y2, the “all of substance Y” refers to Y1 and Y2; the “part of substance Y” refers to Y1 or Y2; or refers to part of Y1 with all of Y2, or, all of Y1 with part of Y2, or, part of Y1 with part of Y2.

The “simultaneous administration” is, e.g., the simultaneous administration of antibody conjugate substance X with “all or part of the substance Y” contained in a separate pharmaceutical composition; or, the simultaneous administration of “a separate pharmaceutical composition containing antibody conjugate substance X” with “a separate pharmaceutical composition comprising all or part of the substance Y”.

The “separate pharmaceutical composition” refers to a single formulation generally accepted in the art for delivering a biologically active compound to a patient (e.g., a mammal).

The “separate administration”, e.g., “separate pharmaceutical composition containing antibody conjugate X” and “separate pharmaceutical composition containing all or part of the substance Y” are administered separately at different times, e.g., one of “separate pharmaceutical composition containing antibody conjugate X” and “separate pharmaceutical composition containing all or part of the substance Y” is administered first, and the other is administered later. The separate administration may be close in time or farther in time.

Whether administered simultaneously or separately, all or some of the administration schemes (including administration route, administration dose, administration interval, etc.) for the antibody conjugate X and substance Y may be the same or different, and they may be adjusted by those skilled in the art according to needs to provide optimal therapeutic effects.

In some embodiments, the antibody conjugate X is administered by injection (e.g., intravenous injection, subcutaneous injection, or intramuscular injection).

In some embodiments, the antibody conjugate X is administered orally.

In some embodiments, all or part of the substance Y is administered by injection (e.g., intravenous injection, subcutaneous injection, or intramuscular injection).

In some embodiments, all or part of the substance Y is administered orally.

In some embodiments, the antibody conjugate X is administered by injection (e.g., intravenous injection, subcutaneous injection, or intramuscular injection); and, all or part of the substance Y is administered by injection (e.g., intravenous injection, subcutaneous injection, or intramuscular injection).

In some embodiments, the antibody conjugate X is administered orally; and, all or part of the substance Y is administered orally.

The present disclosure provides a pharmaceutical composition A comprising the F0002-ADC and a pharmaceutical excipient.

The present disclosure provides a pharmaceutical composition B comprising the pharmaceutical combination and a pharmaceutical excipient.

The pharmaceutical excipients can form a separate pharmaceutical composition together with each component in the pharmaceutical combination, or can form multiple pharmaceutical compositions with each component in the pharmaceutical combination. For example, liposomes can form a separate pharmaceutical composition together with each component in the pharmaceutical composition, and can also form multiple pharmaceutical compositions respectively with each component in the pharmaceutical composition; for another example, liposomes and Doxorubicin hydrochloride form a separate pharmaceutical composition of Doxorubicin hydrochloride liposomes.

Depending on the mode of administration, the pharmaceutical composition can be made in a variety of suitable dosage forms, including gastrointestinal administration dosage forms (e.g., oral dosage forms) and parenteral administration dosage forms (e.g., injection dosage forms).

In some embodiments, the pharmaceutical composition is presented in an oral dosage form.

In some embodiments, the pharmaceutical composition is presented in the form of an injection dosage form (e.g., intravenous injection, subcutaneous injection or intramuscular injection).

The present disclosure provides a use of an antibody conjugate in the preparation of a medicament for the treatment of CD30-positive tumors; in the application, the antibody conjugate is used in combination with the substance Y; the antibody conjugate is the F0002-ADC as described above.

In some embodiments, the CD30-positive tumor is a CD30-positive lymphoma; preferably, the CD30-positive lymphoma is CD30-positive Hodgkin lymphoma, CD30-positive anaplastic large cell lymphoma, CD30-positive diffuse histiocytic lymphoma or CD30-positive cutaneous T cell lymphoma; the most preferably, the CD30-positive lymphoma is CD30-positive Hodgkin lymphoma (its cell is, e.g., CD30-positive Hodgkin lymphoma cells L428 or CD30-positive Hodgkin lymphoma cells L540).

In some embodiments, the CD30-positive tumor is a CD30-positive tumor expressing multidrug resistance gene 1; preferably is CD30-positive Hodgkin lymphoma expressing multidrug resistance gene 1 (its cell is, e.g., CD30-positive Hodgkin lymphoma cells L428 expressing multidrug resistance gene 1 or CD30-positive Hodgkin lymphoma cells L540 expressing multidrug resistance gene 1).

In some embodiments, the CD30-positive tumor is preferably CD30-positive tumor resistant to Adcetris; more preferably, CD30-positive Hodgkin lymphoma resistant to Adcetris (its cell is, e.g., CD30-positive Hodgkin lymphoma cells L428 resistant to Adcetris or CD30-positive Hodgkin lymphoma cells L540 resistant to Adcetris).

On the other hand, the present disclosure provides a method for the treatment of CD30-positive tumors by administering an effective dose of the pharmaceutical composition or the pharmaceutical combination to a patient.

In some embodiments, the CD30-positive tumor is CD30-positive lymphoma; preferably, the CD30-positive lymphoma is CD30-positive Hodgkin lymphoma, CD30-positive anaplastic large cell lymphoma, CD30-positive diffuse histiocytic lymphoma or CD30-positive cutaneous T cell lymphoma; the most preferably, the CD30-positive lymphoma is CD30-positive Hodgkin lymphoma (its cell is, e.g., CD30-positive Hodgkin lymphoma cells L428 or CD30-positive Hodgkin lymphoma cells L540).

In some embodiments, the CD30-positive tumor is CD30-positive tumor expressing multidrug resistance gene 1; preferably CD30-positive Hodgkin lymphoma expressing multidrug resistance gene 1 (its cell is, e.g., CD30-positive Hodgkin lymphoma cells L428 expressing multidrug resistance gene 1 or CD30-positive Hodgkin lymphoma cells L540 expressing multidrug resistance gene 1).

In some embodiments, the CD30-positive tumor is preferably CD30-positive tumor resistant to Adcetris; more preferably, CD30-positive Hodgkin lymphoma resistant to Adcetris (its cell is, e.g., CD30-positive Hodgkin lymphoma cells L428 resistant to Adcetris or CD30-positive Hodgkin lymphoma cells L540 resistant to Adcetris).

In the above application and treatment methods:

The administration schemes (including administration route, administration dose, administration interval, etc.) for the antibody conjugate X and the substance Y may be the same or different, and may be adjusted by those skilled in the art according to needs to provide optimal therapeutic effects.

The antibody conjugate X and all or part of the substance Y may be administered simultaneously or separately.

The antibody conjugate X can be administered by any suitable route in the art, including oral administration, injection (e.g., intravenous, intramuscular, subcutaneous), etc.

In some embodiments, the antibody conjugate X is administered by injection (e.g., intravenous injection, subcutaneous injection, or intramuscular injection).

In some embodiments, the antibody conjugate X is administered orally.

In some embodiments, all or part of the substance Y is administered orally.

In some embodiments, the antibody conjugate X is administered by injection (e.g., intravenous injection, subcutaneous injection, or intramuscular injection); and, all or part of the substance Y is administered orally.

In some embodiments, the antibody conjugate X is administered orally; and, all or part of the substance Y is administered orally.

The present disclosure also provides a preparation method of the antibody conjugate F0002-ADC, which can be referred to as method 1 or method 2 in CN201810078006.9;

The method 1 comprises the following steps:

(i) connecting the linker SMCC with the anti-human CD30 antibody cAC10 to obtain a SMCC-modified antibody;

(ii) connecting the SMCC-modified antibody with DM1 to obtain an antibody conjugate;

The method 2 comprises the following steps:

(i) connecting DM1 with linker SMCC to obtain a connection product;

(ii) connecting the connection product with the anti-human CD30 antibody cAC10 to obtain an antibody conjugate.

In a preferred embodiment of the preparation method of F0002-ADC, the post-treatment of step (i) is: purifying the SMCC-modified antibody.

In a preferred embodiment of the preparation method of F0002-ADC, the post-treatment of step (ii) is: purifying the antibody conjugate.

In a preferred embodiment of the preparation method of F0002-ADC, the purification is gel filtration purification.

In a preferred embodiment of the preparation method of F0002-ADC, the gel filtration purification is carried out by Sephadex G25 purification in step (i) of the method 1 or step (i) of the method 2.

In a preferred embodiment of the preparation method of F0002-ADC, the gel filtration purification is carried out by Superdex 200 purification in step (ii) of the method 1 or step (ii) of the method 2.

In a preferred embodiment of the preparation method of F0002-ADC, the buffer used in the antibody dissolution or purification process contains 50 mM dipotassium hydrogen phosphate-potassium dihydrogen phosphate, 50 mM NaCl and 2 mM EDTA, with a pH of 6.5.

In a preferred embodiment of the preparation method of F0002-ADC, the solvent of SMCC is DMSO or DMA.

In a preferred embodiment of the preparation method of F0002-ADC, the mass molar ratio of the antibody and SMCC used in step (i) of the method 1 is 150-250 mg: 16-34 μmol; and/or, the concentration ratio of the SMCC modified antibody and DM1 used in step (ii) of method 1 is 150-250 mg: 4.8-6.8 μmol; preferably, the mass molar ratio of the antibody and SMCC in step (i) of the method 1 is 200 mg: 30 μmol; and/or, the concentration ratio of the SMCC modified antibody and DM1 used in the step (ii) of the method 1 is 186 mg: 6.8 μmol.

In a preferred embodiment of the preparation method of F0002-ADC, the connection is carried out at 20-30° C.; and/or, in the method 1, the connection time of step (i) is 15 minutes or more, and the connection time of step (ii) is 1-16 hours; preferably, in method 1, the connection time of step (i) is 4 hours, and the connection time of step (ii) is 16 hours.

Purification can be carried out using conventional purification means of the prior art in the field, preferably, gel filtration purification methods are employed in some embodiments of the present disclosure. Gel chromatography is also called molecular sieve filtration, exclusion chromatography, etc. Its outstanding advantages are that the gel used in chromatography belongs to inert carrier, has no charge, weak adsorption force, and the operating conditions is mild, and the process can be carried out in a wide temperature range, does not need organic solvents, and has unique features in maintaining the physical and chemical properties of separated components. It has a good separation effect for high molecular substances. Theoretically, in some embodiments of the present disclosure, a Sephadex G25 column is preferably used; in other embodiments of the present disclosure, preferably, a Superdex 200 chromatographic column is used; the buffer used in gel filtration contains 50 mM dipotassium hydrogen phosphate-potassium dihydrogen phosphate, 50 mM NaCl, 2 mM EDTA, pH 6.5 or other conventional buffers in the field.

To better understand the present disclosure, some terms are defined.

Natural or naturally sequenced CD30 can be isolated from nature or produced by recombinant DNA technology, chemical synthesis, or a combination of the above and similar techniques. Antibody is interpreted in the broadest sense here, which can specifically bind to the target through at least one antigen recognition region located in the variable region of the immunoglobulin molecule, such as carbohydrate, polynucleotide, fat, polypeptide, etc. Specifically, it includes complete monoclonal antibodies, polyclonal antibodies, bispecific antibodies and antibody fragments, as long as they have the required biological activity. The antibodies of the present disclosure can be prepared using techniques well known in the art, such as hybridoma methods, recombinant DNA techniques, phage display techniques, synthetic techniques or combinations thereof, or other techniques known in the art.

Monoclonal antibody means that the antibody comes from a group of basically homogeneous antibodies, that is, the antibodies constituting the cluster are completely identical, except for a few natural mutations that may exist or isomers produced during the preparation of antibody expression. Monoclonal antibodies have a high degree of specificity against a single antigen. In the present disclosure, the monoclonal antibody also specifically includes chimeric antibody and fragments thereof, that is, part of the heavy chain and/or light chain of the antibody comes from a certain, a certain class or a certain subclass, and the rest is related to another, another class or another subclass.

Active fragment of the antibody includes a part of an antibody, optimally an antigen binding region or a variable region. E.g., Fab, part of Fab, Fab2 or dimer form of part of Fab, or even Fv fragment.

Variants of antibodies refer to amino acid sequence mutants, and covalent derivatives of natural peptides, provided that the biological activity equivalent to that of natural polypeptides is retained. The difference between amino acid sequence mutants and natural amino acid sequences is generally that one or more amino acids in the natural amino acid sequence are substituted or one or more amino acids are deleted and/or inserted in the polypeptide sequence. Deletion mutants include fragments of natural polypeptides and N-terminal and/or C-terminal truncation mutants. In the present disclosure, the amino acid sequence mutant has at least 90% or more homology compared with the natural sequence. Homology refers to the percentage of identical amino acid residues after the alignment of amino acid sequences. Methods and procedures for sequence alignment are well known in the art, such as BLAST and Fasta.

Description of the term “drug-antibody ratio” (DAR). L is a group reactive with the conjugation point on the antibody; in the present disclosure, L is SMCC, and the number of L connected to each antibody is represented by m; Y is a cytotoxic agent further conjugated to an antibody linked to L; in the present disclosure, Y is DM1, and the DAR number of each antibody finally conjugated to Y is represented by n. m is greater than or equal to n; in some embodiments, the number of cytotoxic agents conjugated to a single antibody molecule attached to L, i.e., the DAR, is 1, 2, 3, or 4, but due to the specificity of the connection reaction, the DAR of the cytotoxic agent conjugated to the antibody attached to L is actually an average value between 1 and 4, 1 and 3, or 1 and 2, i.e., the antibody conjugate of the present disclosure is actually a mixture of antibodies conjugated to a different number of L-Y or L; in some embodiments; n is an average value between 2 and 4 or 2 and 3; in other embodiments, n is an average value of 1, 2, 3, 4, 5, 6, 7 or 8.

Mertansine alkaloid (DM1) is a thiol-containing mertansine alkaloid derived from the naturally occurring ester ansamitocin P3, which is a common cytotoxic agent. Its chemical name is N2′-Deacetyl-N2′-(3-mercapto-1-oxopropyl)maytansine, and Chemical Abstracts (CAS) number: 139504-50-0. Its molecular formula: C₃₅H₄₈ClN₃O₁₀S; the molecular weight is 738.29. Its structural formula is as follows:

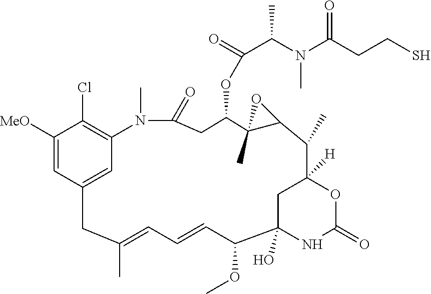

The term “treatment” as used herein refers to therapeutic therapy. When referring to a specific condition, treatment means (1) alleviating one or more biological manifestations of the disease or condition, (2) interfering with (a) one or more points in the biological cascade causing or contributing to the condition or (b) one or more biological manifestations of the condition, (3) ameliorating one or more symptoms, effects, or side effects associated with the condition or its treatment, or one or more symptoms, effects, or side effects, or (4) slowing the development of the condition or one or more biological manifestations of the condition.

The term “treatment” or its equivalent expression, when used for, for example, cancer, refers to a procedure or process for reducing or eliminating the number of cancer cells in a patient or alleviating the symptoms of cancer. The “treatment” of cancer or another proliferative disorder does not necessarily mean that the cancer cells or other disorder will actually be eliminated, but the number of cells or disorder will actually be reduced or the symptoms of cancer or other disorder will actually be alleviated. Generally, the method of treating cancer will be carried out even if it has only a low probability of success, but it is still considered to induce an overall beneficial course of action considering the patient's medical history and estimated survival expectation.

The term “effective dose” used herein refers to an amount of a compound that is sufficient to effectively treat a disease or disorder described herein when administered to a patient. The amount of the compound constituting the “effective dose” will vary according to the compound, the disease and its severity, and the age of the patient to be treated, but it can be adjusted by the person skilled in the art as required.

The term “patient” used herein refers to any animal, preferably a mammal, preferably a human, that is about to receive or has received the compound or composition according to an embodiment of the present disclosure. The term “mammal” used herein includes any mammal. Examples of mammals include, but are not limited to, cattle, horses, sheep, pigs, cats, dogs, mice, rats, rabbits, guinea pigs, monkeys, humans, etc., and humans are most preferred.

The term “pharmaceutical excipient” used herein refers to excipient and additive used in the production of drugs and formulation of prescriptions, and is all substances included in the pharmaceutical preparations except for the active ingredient. Refer to the fourth part of the Pharmacopoeia of the People's Republic of China (2015 edition), or, Handbook of Pharmaceutical Excipients (Raymond C Rowe, 2009 Sixth Edition).

The term “pharmaceutically acceptable” used herein refers to the acids or bases, solvents, excipients, etc., (used in the preparation of the salt) that are generally non-toxic, safe, and suitable for patient use. The “patient” is preferably a mammal, more preferably a human.

The term “pharmaceutically acceptable salt” used herein refers to a salt of a compound prepared with a relatively non-toxic, pharmaceutically acceptable acid or base. When the compound contains relatively acidic functional groups, the base addition salt can be obtained by contacting the neutral form of the compound with a sufficient amount of pharmaceutically acceptable base in a pure solution or a suitable inert solvent. The pharmaceutically acceptable base addition salts include, but are not limited to: lithium, sodium, potassium, calcium, aluminum, magnesium, zinc, bismuth, ammonium, and diethanolamine salts. When the compound contains relatively basic functional groups, the acid addition salt can be obtained by contacting the neutral form of the compound with a sufficient amount of pharmaceutically acceptable acid in a pure solution or a suitable inert solvent. The pharmaceutically acceptable acids include inorganic acids, the inorganic acids include but not limited to: hydrochloric acid, hydrobromic acid, hydroiodic acid, nitric acid, carbonic acid, bicarbonate, phosphoric acid, monohydrogen phosphate, dihydrogen phosphate, phosphorous acid, sulfuric acid, hydrogen sulfate, etc. The pharmaceutically acceptable acids include organic acids, the organic acids include but are not limited to: acetic acid, propionic acid, oxalic acid, isobutyric acid, maleic acid, malonic acid, benzoic acid, succinic acid, octanedioic acid, trans-butenedioic acid, lactic acid, mandelic acid, phthalic acid, benzenesulfonic acid, p-toluenesulfonic acid, citric acid, salicylic acid, tartaric acid, methanesulfonic acid, isonicotinic acid, acid citric acid, oleic acid, tannic acid, pantothenic acid, tartaric acid hydrogen, ascorbic acid, gentianic acid, fumaric acid, gluconic acid, sugar acid, formic acid, ethanesulfonic acid, dihydroxynaphthalic acid (i.e., 4, 4′-methylene-bis(3-hydroxy-2-naphthoic acid)), amino acids (e.g., glutamic acid, arginine), etc. When the compound contains relatively acidic and relatively basic functional groups, it can be converted into a base addition salt or an acid addition salt. For details, refer to Berge et al., “Pharmaceutical Salts”, Journal of Pharmaceutical Science 66: 1-19 (1977), or, Handbook of Pharmaceutical Salts: Properties, Selection, and Use (P. Heinrich Stahl and Camille G. Wermuth, ed., Wiley-VCH, 2002).

The term “solvate” used herein refers to a substance formed by combining a compound of the present disclosure with a stoichiometric or non-stoichiometric solvent. The solvent molecules in the solvate can exist in an ordered or non-ordered arrangement. The solvents include but are not limited to: water, methanol, ethanol, etc.

As used herein, “pharmaceutically acceptable salt” and “solvate” in the term “solvate of pharmaceutically acceptable salt”, as mentioned above, refer to substances formed by the reaction of compounds with relatively nontoxic and pharmaceutically acceptable acids or bases, and combination with stoichiometric or non-stoichiometric solvents.

Unless otherwise stated, the singular forms of “a” or “an” as used herein also include the plural meaning.

The “substance Y”, “pharmaceutically acceptable salt”, “solvate” and “solvate of pharmaceutically acceptable salt” used herein may exist in amorphous or crystalline form. The term “amorphous” refers to the disordered distribution of ions or molecules, i.e., there is no periodic arrangement between ions and molecules. The term “crystalline” means that the ions or molecules in it are arranged in a defined way in a three-dimensional space in a strictly periodic manner, and have a regular pattern of periodic recurrence at a certain distance apart; because of the above differences in periodic arrangement, there can be a variety of crystalline forms, i.e., the phenomenon of polycrystalline forms.

If stereoisomers exist, “substance Y”, “pharmaceutically acceptable salt”, “solvate” and “solvate of pharmaceutically acceptable salt” used herein may exist in the form of a single stereoisomer or a mixture thereof (e. g., racemate). The term “stereoisomer” refers to cis-trans isomer or optical isomer. These stereoisomers can be separated, purified and enriched by asymmetric synthetic methods or chiral separation methods (including but not limited to thin-layer chromatography, rotational chromatography, column chromatography, gas chromatography, high-pressure liquid chromatography, etc.), and can also be obtained by chiral resolution by bonding (chemical bonding, etc.) or salt formation (physical bonding, etc.) with other chiral compounds. The term “single stereoisomer” means that the mass content of a certain stereoisomer in the compound is not less than 95%. A typical single stereoisomer is L-glutamic acid with a purity greater than 98.5%.

If tautomer exists, “Substance Y”, “pharmaceutically acceptable salt”, “solvate” and “solvate of a pharmaceutically acceptable salt” used herein may exist as single tautomers or mixtures thereof, preferably in the form of more stable tautomers. Acetone and 1-propen-2-ol are typical tautomers of each other.

The atoms in “substance Y”, “pharmaceutically acceptable salt” and “solvate” used herein can exist in the form of natural abundance or unnatural abundance. Taking hydrogen atom as an example, its natural abundance refers to that about 99.985% of it is deuterium and about 0.015% is deuterium; its unnatural abundance refers to that about 95% of it is deuterium. That is, one or more of the atoms in “substance Y”, “pharmaceutically acceptable salt”, “solvate” and “solvate of a pharmaceutically acceptable salt” may be an atom in its unnatural abundance. Or, one or more of the atoms in “substance Y”, “pharmaceutically acceptable salt”, “solvate” and “solvate of a pharmaceutically acceptable salt” may be an atom in its natural abundance.

On the basis of not violating common knowledge in the field, the above-mentioned preferred conditions can be combined arbitrarily to obtain preferred embodiments of the present disclosure.

The reagents and raw materials used in the present disclosure are commercially available.

The positive progress effect of the present disclosure is that: the antibody conjugate cAC10-SMCC-DM1 (hereinafter referred to as F0002-ADC) shows high killing activity against a variety of CD30-positive tumor cells, including Karpas299, L540, HH, L428, etc., which has therapeutic value for CD30-positive HL, ALCL, CTCL, etc. The F0002-ADC has a strong killing effect on HL cells L428 cells in vitro, and it is expected to obtain better clinical effect for tumors insensitive to Adcetris (brentuximab vedotin). And compared with Adcetris, F0002-ADC has significantly improved non-clinical safety, has a larger safety window, and is expected to become a safer drug for the treatment of CD30-positive tumors.

F0002-ADC enters tumor cells through endocytosis in the cell membrane of CD30, and enzymatically releases the main active substance Lys-MCC-DM1 in the lysosome, which can inhibit the formation of tubulin, inhibit mitosis, and induce cell apoptosis, while Lys-MCC-DM1 released by cell lysis does not pass well through the cell membrane and does not have “bystander effect” (Blood. 2016; 128(12):1562-6), and only specifically kills CD30-positive tumor cells, avoiding the damage to normal cells.

### DETAILED DESCRIPTION OF THE PREFERRED EMBODIMENT

The present disclosure will be further explained by way of embodiments below, but the present disclosure is not limited to the scope of the described embodiments. Experimental methods for which specific conditions are not indicated in the following embodiments are selected according to conventional methods and conditions, or according to the commercial specification.

### Embodiment 1 Construction and Expression of Anti-Human CD30 Antibody cAC10

The fully synthesized DNA fragments encoding the heavy and light chains of cAC10 monoclonal antibody were (see U.S. Pat. No. 7,090,843, B1RECOMBINANT ANTI-CD30 ANTIBODIES AND USES THEREOF, Seattle Genetics, Inc., 2006, SEQ ID NO:1 and SEQ ID No:9; and International Nonproprietary Names for Pharmaceutical Substances (INN). WHO Drug Information Vol. 24, No. 2, 2010) cloned into the pEE12.4 eukaryotic expression vector of Lonza Company, respectively, and the enzymatic cleavage and ligation were performed according to the instructions of the commercially available kit (DNA Ligation kit Ver2.0, TAKARA).

The constructed heavy chain and light chain expression vectors were transformed into E. coli DH5a respectively, and the positive clones were selected and inoculated in 500 mL LB medium for amplification. DNA was extracted and purified using Qiagen's Ultrapure Plasmid DNA Purification Kit according to the manufacturer's instructions. The above plasmid DNA containing heavy and light chain coding sequences was co-transfected into CHO-K1 (Chinese hamster ovary cells, purchased from ATCC) in a certain ratio using Invitrogen's liposome method kit, and the procedure was performed according to the manufacturer's instructions.

The DMEM/F12 (1:1) cell medium (Invitrogen) was replaced with GMEM screening medium (Sigma) containing the screening drug 24-48 hours after transfection, and the screening medium was changed every 3-4 days until cell clone formation. When the diameter of the cell clone reached 1 mm-2 mm, the monoclonal was picked from the plate by clone ring and transferred to a 24-well plate containing 1 mL of screening medium. When single cell clones grew to 50%-70% full layer in 24-well plates, the culture supernatant of each clone was taken for ELISA detection, and the cell clones with high expression were selected for drug-pressurized amplification screening. When the concentration of the screened drugs rose to the highest level, the expression level of each cloned single cell was detected, and cells with high expression levels and good cell growth status were selected for amplification culture. Recombinant cell culture supernatant was collected and purified by protein A affinity chromatography for functional evaluation.

Recombinant human CD30 antigen (coating antigen, R&D Systems) was diluted to a certain concentration (0.5 μg/mL) with a coating solution (pH 9.6 CBS) and coated on 96-well plates, 100 μL/well, and placed at 2° C.-8° C. overnight. The liquid in the hole was discarded, then the mixture was washed with PBST for 3 times, dried by spin, and then added with 400 μL/hole sealing solution (1% BSA PBST) at room temperature for 2 hours, then washed with PBST for 3 times and spin-dried. The standard was diluted with diluent to a certain concentration, and the expression supernatant was diluted appropriately according to the situation, the sample was added to a 96-well plate with 100 μL/well replicate, incubated at 37° C. for 1 hour, the liquid was discarded, the plate was washed 3 times, and spin-dried. Goat anti-human IgG(Fc)-HRP (enzyme-linked antibody, PIERCE company) was diluted with diluent at a ratio of 1:20,000, added to a 96-well plate at 100 μL/well, the reaction was carried out at 37° C. for 1 hour, the liquid was discarded, the plate was washed 3-6 times, and spin-dried. The substrate mixture was prepared, added into a 96-well plate at 100 μL/well, and incubated at 37° C. for 20 min. 100 μL/well of stopping solution was added to stop the reaction. The wavelength of 655 nm was used as the reference wavelength, and the absorbance was measured at 450 nm; the content of the samples was calculated according to the standard curve, and the clones with high expression were selected for amplification and culture. Recombinant cell culture supernatant was collected and purified by protein A affinity chromatography for subsequent ligation experiments.

### Embodiment 2 Detection Method of DAR

DAR detection was based on the UV absorption of Ab and DM1, and the coupling degree was calculated by measuring the absorbance at 252 nm and 280 nm, the average number of DM1 connected to each antibody molecule was determined. Ultraviolet/visible spectrophotometry (UV/Vis) was a simple and convenient method, which can be used to determine the protein concentration and the average number of drugs conjugated by the antibody in the antibody-drug conjugate (ADC). DAR can be determined by using ADC absorbance measurements and extinction coefficients of corresponding antibodies and drugs.

When the absorption coefficient of a pure substance under certain conditions was known, the tested sample can be prepared into a solution under the same conditions, and its absorbance was measured, and the content of the substance can be calculated by the following formula: A=E×c×1, wherein A is absorbance, E is absorption coefficient, c is protein content, and 1 is liquid layer thickness (cm). This formula is also applicable to multi-component systems; if these components have different absorption spectra and there is no interaction between them, the light absorption of these components in the sample solution can be added. At this time, the absorbance Aλ=(E1λ×C1+E2λ×C2+ . . . +Enλ×cn)×1, n is the number of different absorption components, and Enλ is the extinction coefficient of the nth component; cn is the concentration of the nth component.

The F0002-ADC sample is known to have chromophores in the ultraviolet region, and the cAC10 monoclonal antibody (labeled as mab in the following formula) has an obvious maximum absorption value at 280 nm±3 nm, while the drug (DM1, labeled as a drug in the following formula) has a maximum absorption value at 252 nm±3 nm, and the presence of a drug does not affect the light absorption properties of the antibody. Therefore, by applying the above formula:

A₂₈₀=(E_{drug}²⁸⁰×C_{drug}+E_{mab}²⁸⁰×C_{mab})×l

A₂₅₂=(E_{drug}²⁵²×C_{drug}+E_{mab}²⁵²×C_{mab})×l

to reduce the systematic error, the value of the reference wavelength A₃₂₀ was subtracted from the measured values of both A₂₈₀ and A₂₅₂, and then A₂₈₀ and A₂₅₂ were substituted into the above equation and combined with the two equations above to obtain the concentrations of antibody and drug.

$${C}_{mab} = \frac{{A}_{280 \times } {E}_{drug}^{252} - {A}_{252 \times } {E}_{drug}^{280}}{( {E}_{mab}^{280} {E}_{drug}^{252} - {E}_{mab}^{252} {E}_{drug}^{280} ) \times l}$$

$${C}_{durg} = \frac{{A}_{280 \times } {E}_{mab}^{252} - {A}_{252 \times } {E}_{mab}^{280}}{( {E}_{drug}^{280} {E}_{mab}^{252} - {E}_{drug}^{252} {E}_{mab}^{280} ) \times l}$$

Then the average drug antibody coupling ratio (DAR) was calculated as follows:

$$DAR = \frac{{C}_{drug}}{{C}_{mab}}$$

E_{drug}²⁸⁰=5700 L/mol·cm; E_{drug}²⁵²=26790 L/mol·cm;

E_{mab}²⁸⁰=235454 L/mol·cm; E_{mab}²⁵²=83816 L/mol·cm.

### Embodiment 3 Preparation of Antibody Conjugate

The present disclosure ensured the stability of the average coupling ratio (DAR) of the toxic antibody by controlling the feeding ratio, pH, temperature and stirring of the coupling reaction.

A. Linker SMCC Modified Antibody:

Configuration of buffer: 50 mM dipotassium hydrogen phosphate-potassium dihydrogen phosphate, 50 mM NaCl, 2 mM EDTA, pH 6.5. The medium of the antibody was changed with buffer by 10 times volume ultrafiltration, and the final concentration of the antibody was 10 mg/mL, argon gas was added until it was full. 1.5 mL of 20 mM concentration of SMCC (dissolved in DMA) was added to 20 mL of cAC10 monoclonal antibody (Brentuximab, i.e., brentuximab) solution and the reaction was carried out for 4 hours at room temperature. The reaction mixture was filtered by a Sephadex G25 gel column, and the column was first equilibrated with buffer at 5 times the column volume. The characteristic peaks of the antibodies were collected at OD280 to obtain the antibodies modified by SMCC.

B. Coupling of Modified Antibodies with Mertansine (DM1):

The antibody modified by SMCC was diluted to a final concentration of 3 mg/mL with buffer for a total of 62 mL. Then, 1.7 mL of DM1 solution dissolved in DMA (concentration was 4.0 mM) was added to the antibody diluent. The reaction was carried out at room temperature (20° C.-30° C.) for 16 hours under the protection of argon. The reaction solution was chromatographed by Superdex 200, and the characteristic peak of the antibody was collected under OD280 to obtain the target product.

The structural formula of the obtained F0002-ADC antibody conjugate was (wherein mAb was cAC10 monoclonal antibody):

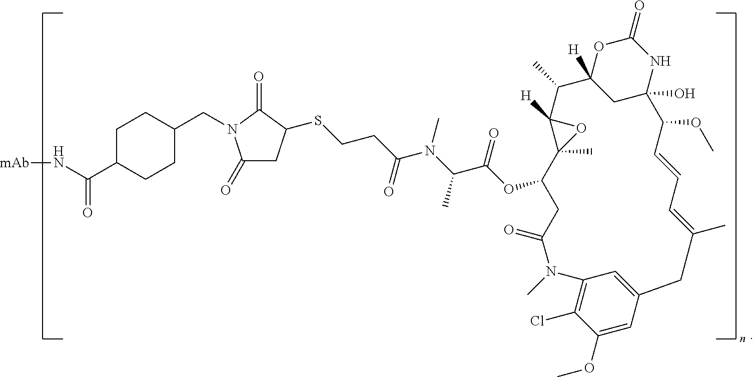

According to the detection method in embodiment 2, the DAR of the antibody conjugate obtained in this embodiment was 3.6. The distribution of their different DAR values by LC-MS analysis was as follows:

| DAR | D0 | D1 | D2 | D3 | D4 | D5 | D6 | D7 |
|---|---|---|---|---|---|---|---|---|
| | 3% | 10% | 17% | 20% | 18% | 16% | 9% | 7% |

3.6

The proportions of different DAR distributions were obtained by LC-MS and then multiplied by the corresponding DAR values, and finally summed to obtain an average DAR value of 3.6.

### Effect Embodiment 1 In Vitro Killing

Human degenerative large cell lymphoma cells Karpas299 (Nanjing Cobioer) were seeded at 5×10⁴ cells/mL, and serially diluted samples of the antibody conjugate prepared above, cAC10 monoclonal antibody and commercially available Adcetris were added. After the sample was added, it was cultured in 5% CO₂ at 37° C. for 77±2 hours, stained with AlamarBlue fluorescent dye, cultured for 19±2 hours, and read at 530 nm (excitation)/590 nm (emission). The IC₅₀ value of the semi-inhibitory concentration of each sample was calculated by fitting with the computer's four-parameter equation software. The assay results of Karpas299 showed that, with the increase of the concentration of F0002-ADC, the survival rate of tumor cells decreased significantly, showing a strong cell killing effect. However, cAC10 monoclonal antibody had basically no inhibitory effect on cell growth. As a comparative embodiment, the killing activity of Adcetris was equivalent to that of F0002-ADC.

TABLE 1
Summary table of cell survival rate of Karpas299 cells from different
samples

| | | F0002 monoclonal | | | |
|---|---|---|---|---|---|
| F0002-ADC | | antibody | | Adcetris | |
| | Cell survival | | Cell survival | | Cell survival |
| Concentration | rate | Concentration | rate | Concentration | rate |
| (ng/mL) | (%) | (ng/mL) | (%) | (ng/mL) | (%) |
| 50 | 27 | 50 | 92 | 50 | 24 |
| 25 | 28 | 25 | 93 | 25 | 28 |
| 12.5 | 34 | 12.5 | 97 | 12.5 | 39 |
| 6.25 | 51 | 6.25 | 96 | 6.25 | 70 |
| 4.17 | 76 | 4.17 | 96 | 4.17 | 82 |
| 2.78 | 87 | 2.78 | 98 | 2.78 | 91 |
| 1.85 | 90 | 1.85 | 99 | 1.85 | 93 |
| 0.74 | 96 | 0.74 | 100 | 0.74 | 97 |
| 0.30 | 96 | 0.30 | 100 | 0.30 | 98 |

Furthermore, compared to Adcetris, the killing activity of F0002-ADC in different CD30-positive tumor cells, such as Hodgkin lymphoma cells L428, cutaneous somatic lymphoma HH cells, Hodgkin lymphoma cells L540 and human degenerative large cell lymphoma cells SU-DHL-1, was measured and characterized by IC₅₀ value, the results were shown in table 2.

TABLE 2
Comparison of killing activity in vitro against different
CD30-positive tumor cells

| IC₅₀ (ng/mL) | Karpas299 | L428 | HH | L540 | SU-DHL-1 |
|---|---|---|---|---|---|
| F0002-ADC | 10.6 | 5.7 | 3.2 | 8.6 | 8.1 |
| adcetris | 14.8 | 54314 | 3.2 | 5.9 | 12.5 |

As shown in table 2, wherein, Adcetris showed significant resistance to Hodgkin lymphoma cells L428 cells with a IC₅₀ value up to 54,314 ng/mL, which may be related to the clinical insensitivity of Adcetris in some patients. In contrast, F0002-ADC showed significant killing activity against L428 cells.

It is known in the art that small molecule sensitivity does not mean that the ADC drug coupled to the antibody still has sensitive activity. For example, the small molecule MMAE in adcetris is sensitive to L428, but adcetris is not sensitive to L428. In the present disclosure, the antibody of F0002-ADC is the same as that of adcetris, but the difference lies in the linker and cytotoxic agent; however, during the project development, we unexpectedly found that F0002-ADC is sensitive to L428 and has significant killing activity through the above experiments; the unexpected results were obtained.

### Effect Embodiment 2 Identification of MDR1 Expression

The reason for drug resistance of Hodgkin lymphoma cells L428 is that there is no accumulation of MMAE in cells, which leads to not enough active small molecules to kill tumor cells, this may be related to the expression of drug resistance pump in this cell; the expression level of multidrug resistance pump on the surface of different CD30 tumor cells was analyzed by flow cytometry. The sample was taken for 1×10⁶ cells/tube, centrifuged at 100 g for 5 min, washed once with 1 mL PBS, added with 100 μL PBS to resuspend the cells, then added with 5 μL/tube of CD243 (ABC B1) monoclonal antibody (UIC 2) and PE antibody (ThermoFisher), then 5 μL/tube of PBS was added to the negative control, and the cells were incubated on ice for 30 min, washed twice with cold PBS. The expression level of MDR1 was evaluated by flow cytometry after resuspension of cells in 0.2 mL PBS/tube to detect the fluorescence signal. The results showed that L428 cells specifically expressed MDR1 on the surface, while other CD30 cells did not express MDR 1; with the addition of 12.5 μg/mL Verapamil, the killing effect of Adcetris on L428 can reach the activity level equivalent to that of F0002-ADC. It was demonstrated that the main reason for Adcetris resistance in L428 cells was attributed to the high expression of MDR1.

TABLE 3
expression level of MDR1 in different cell lines

| Cells | MDR1 binding ratio |
|---|---|
| HH | 2.6 |
| Karpas 299 | 1.2 |
| L540 | 1.2 |
| L428 | 16.1 |

TABLE 4
effect of MDR1 inhibitor on the killing effect of Adcetris on L428

| | | Adcetris + | | | |
|---|---|---|---|---|---|
| F0002-ADC | | Verapamil (12.5 μg/mL) | | Adcetris | |
| Concentration | Cell survival | Concentration | Cell survival | Concentration | Cell survival |
| (ng/mL) | rate (%) | (ng/mL) | rate (%) | (ng/mL) | rate (%) |
| 500 | 36 | 500 | 16 | 500000 | 30 |
| 20 | 38 | 20 | 36 | 50000 | 32 |
| 8 | 40 | 8 | 89 | 5000 | 41 |
| 3.20 | 46 | 3.20 | 94 | 500 | 90 |
| 1.28 | 48 | 1.28 | 96 | 50 | 100 |
| 0.51 | 67 | 0.51 | 100 | 5 | 102 |
| 0.20 | 86 | 0.20 | 100 | 0.50 | 100 |
| 0.08 | 100 | 0.08 | 99 | 0.05 | 100 |
| 0.03 | 102 | 0.03 | 100 | 0.01 | 100 |

### Effect Embodiment 3 Adcetris Induced Drug-Resistant Cell Lines

A concentration gradient increasing, pulsating method was employed, i.e., the cell density was 2.5×10⁴/mL, the killing effect time was chosen to be 4 days, the recovery culture time was 3 days, and the concentration gradient of Adcetris was chosen to be 1×IC₅₀, 2×IC₅₀, 5×IC₅₀, 10×IC₅₀, with IC₅₀ referring to the previous cytotoxicity results. The Adcetris resistant cell lines Karpas299, L428, HH, L540 and SU-DHL-1 were screened, and the drug resistant cell lines Karpas299-R, L428-R, HH-R, L540-R and SU-DHL-1-R were screened out respectively.

### Effect Embodiment 4 Killing Evaluation of Drug Resistant Cells

The killing activity of F0002-ADC compared to Adcetris was evaluated separately for the above drug-resistant cell lines treated with Adcetris induction and characterized by IC₅₀ values. As shown in Table 6 below, HH, Karpas299, L540 and SU-DHL-1 drug-resistant cells induced by Adcetris had significant drug-resistant activity to Adcetris. Further analysis showed that the drug resistance of HH, Karpas299 and SU-DHL-1 cells was mainly caused by the down-regulation of CD30 antigen abundance, and they also had drug resistance activity to F0002-ADC. The drug resistance of L540 cells to Adcetris was mainly caused by the high expression of MDR1, and its mRNA was increased by 6.3 times compared with before drug resistance. The results showed that F0002-ADC still had sensitive killing activity against L540 cells after drug resistance, indicating that F0002-ADC had specific and sensitive killing activity against the occurrence of MDR1 high expression after treatment with Adcetris.

TABLE 5
sensitivity of cells to Adcetris before and after L540 drug resistance

| L540 cells | | | | L540-R cells | | | |
|---|---|---|---|---|---|---|---|
| F0002-ADC | | Adcetris | | F0002-ADC | | Adcetris | |
| | Cell | | Cell | | Cell | | Cell |
| Concentration | survival | Concentration | survival | Concentration | survival | Concentration | survival |
| (ng/mL) | rate (%) | (ng/mL) | rate (%) | (ng/mL) | rate (%) | (ng/mL) | rate (%) |
| 50 | 22 | 50 | 30 | 500000 | 15 | 500000 | 17 |
| 25 | 34 | 25 | 34 | 250000 | 13 | 250000 | 18 |
| 12.5 | 42 | 12.5 | 50 | 125000 | 14 | 125000 | 19 |
| 8.33 | 65 | 8.33 | 65 | 62500 | 15 | 62500 | 26 |
| 5.56 | 73 | 5.56 | 78 | 31250 | 16 | 31250 | 34 |
| 3.70 | 78 | 3.70 | 88 | 15625 | 17 | 15625 | 39 |
| 1.48 | 78 | 1.48 | 92 | 7812.5 | 19 | 7812 | 46 |
| 0.59 | 78 | 0.59 | 94 | 3906.2 | 20 | 3906 | 47 |
| 0.24 | 76 | 0.24 | 95 | 1953.1 | 19 | 1953 | 47 |
| / | | | | 976.6 | 20 | 976.6 | 51 |
| | | | | 488.3 | 20 | 488.3 | 50 |
| | | | | 244.1 | 20 | 244.1 | 49 |
| | | | | 122.1 | 21 | 122.1 | 53 |
| | | | | 61.04 | 20 | 61.04 | 51 |
| | | | | 30.52 | 20 | 30.52 | 55 |
| | | | | 15.26 | 23 | 15.26 | 58 |
| | | | | 7.63 | 46 | 7.63 | 65 |
| | | | | 3.81 | 72 | 3.81 | 72 |

TABLE 6
comparison of antigen abundance, MDR1 level and killing activity of
different tumor cells before and after drug resistance

| | CD30 binding | IC₅₀ (ng/mL) | | MDR1 |
|---|---|---|---|---|
| Cells | ratio | F0002-ADC | Adcetris | mRNA |
| HH | 254 | 1.5 | 1.5 | Not detected |
| HH-R | 48 | 3921 | 36714 | Not detected |
| Karpas 299 | 112 | 3.5 | 4.4 | Not detected |
| Karpas 299-R | 16 | 249 | 63.7 | Not detected |
| SU-DHL-1 | 68 | 9.8 | 7.0 | Not detected |
| SU-DHL-1-R | 13 | 891 | 63.7 | Not detected |
| L540 | 104 | 10.9 | 8.8 | 1 |
| L540-R | 42 | 8.2 | 3053 | 6.3 |
| L428 | 48 | 2.9 | 27157 | 8.2 |

Note:
CD30 binding ratio indicates the ratio of CD30 flow signal with negative cell romas.

### Effect Embodiment 5 Verification of In Vivo System Model

The above embodiments confirmed that F0002-ADC maintains killing sensitive activity on tumor cells with high MDR1 expression, and has good tumor killing inhibitory activity on cells with high MDR1 expression induced by Adcetris treatment, indicating that F0002-ADC can specifically overcome MDR1 drug resistance; the purpose of this embodiment is to further verify the anti-tumor efficacy of F0002-ADC and Adcetris on the in vivo system model of L428 cells expressing MDR1.

To establish the L428 in vivo systemic model, 11 to 12 week-old female NPG mice were injected with 1×10⁷ human lymphoma cells (L428) dissolved in 100 μL of PBS solution via tail vein. On the 8th day after cell inoculation, the mice were randomly divided into three treatment groups, with 6 mice in each group, namely blank control group/F0002-ADC group (3 mg/kg)/Adcetris group (3 mg/kg). The first dosing was started on the day of grouping (D8), F0002-ADC and Adcetris were prepared into 2.5 mL target solutions with a concentration of 0.6 mg/mL, the dosing method was tail vein administration, the dosing cycle was Q3W*2 (D8 and D29), the body weight of the experimental animals was measured twice a week and the status of the experimental animals was observed, after the end of the second dosing, the status of the experimental animals was closely monitored; and mice with deteriorating physical condition, near death or unable to feed and drink normally were euthanized, until all animals in the treated group died due to disease progression; after the experiment, SPSS was used for statistical analysis, and Kaplan-Meier method was used to draw the survival curve of each group. Compared with the blank control group, both F0002-ADC and Adcetris significantly prolonged the survival period of the lymphoma model mice, the blank group showed animal death from D53 after cell inoculation, and all the mice in the group died on D79; and the F0002-ADC group showed animal death from D66 after cell inoculation, and all the mice in the group died on D114; the mice in the Adcetris group showed animal death from D58 after cell inoculation, and all the mice in the group died on D90. The F0002-ADC group (3 mg/kg) had a good effect on prolonging the survival of human lymphoma model mice compared with the blank control group, P<0.001 was statistically different and highly significant; the Adcetris group (3 mg/kg) had a certain effect on prolonging the survival of model mice compared with the blank control group, but P>0.05 was not statistically different. There was a statistical difference between the F0002-ADC group and the Adcetris group (p<0.05).

TABLE 7
comparison of survival time

| Days/survival rate | Blank control (%) | F0002-ADC (%) | Adcetris (%) |
|---|---|---|---|
| 0 | 100 | 100 | 100 |
| 53 | 83 | 100 | 100 |
| 57 | 83 | 100 | 100 |
| 58 | 83 | 100 | 83 |
| 60 | 83 | 100 | 67 |
| 64 | 50 | 100 | 67 |
| 66 | 33 | 83 | 67 |
| 68 | 33 | 83 | 50 |
| 73 | 33 | 83 | 33 |
| 75 | 17 | 83 | 33 |
| 79 | 0 | 83 | 33 |
| 85 | 0 | 67 | 33 |
| 87 | 0 | 50 | 33 |
| 90 | 0 | 50 | 0 |
| 94 | 0 | 33 | 0 |
| 114 | 0 | 0 | 0 |

### Effect Embodiment 6 Combined ABVD/AVD Scheme

Cell proliferation method was used to investigate the killing effect of drugs on cells. L428 cells were inoculated in 96-well cell culture plates at 5×10⁴ cell/mL, i.e., 5000 cells per well. Doxorubicin was diluted to 259.5 ng/mL, 129.8 ng/mL, 64.88 ng/mL, 32.44 ng/mL, 21.63 ng/mL, 14.42 ng/mL, 9.611 ng/mL, 3.844 ng/mL, 1.538 ng/mL using cell culture medium, for a total of 9 concentrations. Serial dilutions of Doxorubicin were added to the culture plates of the seeded plates and incubated for 77±2 hours at 37° C. in a 5% CO₂ incubator. The samples were stained with AlamarBlue fluorescent dye and incubated at 37° C. in a 5% CO₂ incubator for 19±2 hours. Readings were performed at wavelength of 50 nm (excitation)/590 nm (emission). The IC₅₀ value of Doxorubicin was calculated by fitting it with four-parameter equation.

The killing curves of four single drugs of ABVD and F0002-ADC against L428 and L540 and IC₅₀ values of single drug were obtained by the same method described above, wherein the dose-effect concentration range of the drugs could be obtained by pre-experiments.

According to Hodgkin lymphoma drug combination scheme, the classic combination mode of ABVD was selected first, and the combination effect of F0002-ADC with ABVD or AVD scheme was studied. Single drug killing was first performed: single drug experiments were performed on F0002-ADC, Doxorubicin, Bleomycin, Vincristine and Dacarbazine against L428 and L540 cells, respectively, to obtain the IC₅₀ values of inhibitory killing at different dose concentrations, as shown in Table 8 below.

TABLE 8
summary table of IC₅₀ value of each cell of single drug

| Drug name | L428 (IC₅₀, nM) | L540 (IC₅₀, nM) |
|---|---|---|
| F0002-ADC | 0.075 | 0.070 |
| Doxorubicin | 27.79 | 58.49 |
| Bleomycin | 2043 | 3198 |
| Vincristine | 28.08 | 0.772 |
| Dacarbazine | 178140 | 31346 |

The combination method was chosen as a constant combination ratio (i.e., the ratio of IC₅₀), and F0002-ADC was combined with ABVD or AVD for killing experiments against L428 and L540 cells. Calcusyn, the software for calculating the combination index, was chosen to calculate the CI of the combination index at different killing levels,

$$CI = {\sum }_{i = 1}^{n} \frac{( D ) i}{( {D}_{x} ) j} ,$$

wherein D is the single dose and Dx is the combination dose. CI value less than 1 was synergistic, equal to 1 was additive, and greater than 1 was antagonistic.

(1) In the combination scheme of F0002-ADC+ABVD (Doxorubicin, Bleomycin, Vincristine and Dacarbazine), the dosing molar ratio of F0002-ADC:Doxorubicin:Bleomycin:Vincristine:Dacarbazine was 1:400:30000:400:3000000; L428 cells were treated with multiple concentrations for 96 hours, in order to investigate the killing results of ABVD scheme combined with F0002-ADC, and compared with F0002-ADC; the combined index CI value was less than 1 when the cell killing rate was 500%, 7500 and 900%; the results showed that F0002-ADC had synergistic effect after combined with AB VD, and could obtain a better killing effect.

TABLE 9
F0002-ADC combined with ABVD in L428 cells

| | | F0002-ADC + ABVD | | | | | |
|---|---|---|---|---|---|---|---|
| | | | A | B | V | D | |
| F0002-ADC | | F0002-ADC | (Doxorubicin) | (Bleomycin) | (Vincristine) | (Dacarbazine) | |
| Concentration | Cell survival | Concentration | Concentration | Concentration | Concentration | Concentration | Cell survival |
| (nM) | rate (%) | (nM) | (nM) | (μM) | (nM) | (μM) | rate (%) |
| 0.33 | 29 | 0.066 | 26.40 | 1.98 | 26.40 | 198 | 17 |
| 0.17 | 33 | 0.034 | 13.60 | 1.02 | 13.60 | 102 | 19 |
| 0.08 | 35 | 0.016 | 6.40 | 0.48 | 6.40 | 48 | 20 |
| 0.04 | 46 | 0.008 | 3.20 | 0.24 | 3.20 | 24 | 28 |
| 0.03 | 65 | 0.006 | 2.40 | 0.18 | 2.40 | 18 | 44 |
| 0.02 | 79 | 0.004 | 1.60 | 0.12 | 1.60 | 12 | 73 |
| 0.01 | 87 | 0.002 | 0.80 | 0.06 | 0.80 | 6 | 82 |
| 0.003 | 88 | 0.001 | 0.24 | 0.02 | 0.24 | 1.80 | 89 |

(2) Also in L540 cells, in the combination scheme of F0002-ADC+ABVD, the molar ratio of F0002-ADC:Doxorubicin:Bleomycin:Vincristine:Dacarbazine was 1:800:45000:11:550000; the cells were treated with multiple concentrations for 96 hours, and the combined index CI value obtained by calculation was less than 1 when the cell killing rate was 5000, 7500 and 900%; the results showed that F0002-ADC had synergistic effect after combined with ABVD, and could obtain better killing effect.

TABLE 10
F0002-ADC combined with ABVD in L540 cells

| | | F0002-ADC + ABVD | | | | | |
|---|---|---|---|---|---|---|---|
| | | | A | B | V | D | |
| F0002-ADC | | F0002-ADC | (Doxorubicin) | (Bleomycin) | (Vincristine) | (Dacarbazine) | |
| Concentration | Cell survival | Concentration | Concentration | Concentration | Concentration | Concentration | Cell survival |
| (nM) | rate (%) | (nM) | (nM) | (μM) | (nM) | (μM) | rate (%) |
| 0.33 | 27 | 0.066 | 52.80 | 2.97 | 0.73 | 36.30 | 14 |
| 0.17 | 43 | 0.034 | 27.20 | 1.53 | 0.37 | 18.70 | 27 |
| 0.08 | 58 | 0.016 | 12.80 | 0.72 | 0.18 | 8.80 | 33 |
| 0.04 | 66 | 0.008 | 6.40 | 0.36 | 0.09 | 4.40 | 44 |
| 0.03 | 69 | 0.006 | 4.80 | 0.27 | 0.07 | 3.30 | 58 |
| 0.02 | 77 | 0.004 | 3.20 | 0.18 | 0.04 | 2.20 | 73 |
| 0.01 | 84 | 0.002 | 1.60 | 0.09 | 0.02 | 1.10 | 90 |
| 0.003 | 97 | 0.001 | 0.48 | 0.03 | 0.01 | 0.33 | 93 |

(3) In the combination scheme of F0002-ADC+AVD (Doxorubicin, Vincristine and Dacarbazine), the dosing molar ratio of F0002-ADC:Doxorubicin:Vincristine:Dacarbazine was 1:400:400:3000000; L428 cells were treated with multiple concentrations to examine the killing results of the AVD scheme combined with F0002-ADC. The combined index CI value obtained by calculation was less than 1 when the cell killing rate was 500%, 7500 and 900%; the results showed that F0002-ADC had synergistic effect after combined with AVD, and could obtain better killing effect.

TABLE 11
F0002-ADC combined with AVD in L428 cells

| | | F0002-ADC + AVD | | | | |
|---|---|---|---|---|---|---|
| | | | A | V | D | |
| F0002-ADC | | F0002-ADC | (Doxorubicin) | (Vincristine) | (Dacarbazine) | |
| Concentration | Cell survival | Concentration | Concentration | Concentration | Concentration | Cell survival |
| (nM) | rate (%) | (nM) | (nM) | (nM) | (μM) | rate (%) |
| 0.33 | 29 | 0.086 | 33 | 33 | 247.5 | 17 |
| 0.17 | 33 | 0.042 | 17 | 17 | 127.5 | 19 |
| 0.08 | 35 | 0.020 | 8 | 8 | 60 | 20 |
| 0.04 | 46 | 0.010 | 4 | 4 | 30 | 37 |
| 0.03 | 65 | 0.008 | 3 | 3 | 22.50 | 54 |
| 0.02 | 79 | 0.005 | 2 | 2 | 15 | 68 |
| 0.01 | 87 | 0.002 | 1 | 1 | 7.50 | 68 |
| 0.003 | 88 | 0.001 | 0.30 | 0.30 | 2.25 | 69 |

(4) In the combination scheme of F0002-ADC+AVD, the dosing molar ratio of F0002-ADC:Doxorubicin:Vincristine:Dacarbazine was 1:800:11:550000; cells were treated with multiple concentrations in order to investigate the killing results of AVD scheme combined with F0002-ADC, and the combined index CI value obtained by calculation was less than 1 at 500%, 7500 and 900% of cell killing; the results indicated that F0002-ADC had a synergistic effect after combining with AVD, and could obtain a better killing effect.

TABLE 12
F0002-ADC combined with AVD in L540 cells

| | | F0002-ADC + AVD | | | | |
|---|---|---|---|---|---|---|
| | | | A | V | D | |
| F0002-ADC | | F0002-ADC | (Doxorubicin) | (Vincristine) | (Dacarbazine) | |
| Concentration | Cell survival | Concentration | Concentration | Concentration | Concentration | Cell survival |
| (nM) | rate (%) | (nM) | (nM) | (nM) | (μM) | rate (%) |
| 0.33 | 27 | 0.082 | 66 | 0.91 | 45.38 | 16 |
| 0.17 | 43 | 0.042 | 34 | 0.47 | 23.38 | 23 |
| 0.08 | 58 | 0.020 | 16 | 0.22 | 11 | 33 |
| 0.04 | 66 | 0.010 | 8 | 0.11 | 5.50 | 43 |
| 0.03 | 69 | 0.008 | 6 | 0.08 | 4.12 | 60 |
| 0.02 | 77 | 0.005 | 4 | 0.06 | 2.75 | 79 |
| 0.01 | 84 | 0.002 | 2 | 0.03 | 1.39 | 80 |
| 0.003 | 97 | 0.001 | 0.60 | 0.01 | 0.41 | 88 |

### Effect Embodiment 7 Other Combination Schemes

A single drug scheme was used to study the inhibitory effects of Bleomycin, Etoposide, Doxorubicin, Cyclophosphamide, Vincristine, Procarbazine and Prednisone, etc., on L428, L540 cells and Karpas299 cells; the IC₅₀ values were shown in the table below, and then the drugs were combined to kill, and constant combination ratio was used in the combination method, i.e., IC₅₀ ratio. See table 13 below.

TABLE 13
summary table of IC₅₀ value of each cell of single drug

| | L428 cells | L540 cells | Karpas299 cells |
|---|---|---|---|
| Drug name | (IC₅₀, nM) | (IC₅₀, nM) | (IC₅₀, nM) |
| F0002-ADC | 0.075 | 0.070 | 0.033 |
| Bleomycin | 2043 | 3198 | 1680 |
| Etoposide | 54160 | 30089 | 64111 |
| Doxorubicin | 27.79 | 58.49 | 40.51 |
| Cyclophosphamide | 590444 | 120550 | 385446 |
| Vincristine | 28.08 | 0.772 | 18.54 |
| Procarbazine | 500438 | 24689 | 26899 |
| Prednisone | 112854 | 98544 | 186715 |

(1) In the combination scheme of F0002-ADC+BEACOPP (Bleomycin, Etoposide, Doxorubicin, Cyclophosphamide, Vincristine, Procarbazine and Prednisone), the dosing molar ratio of F0002-ADC:Bleomycin:Etoposide:Doxorubicin:Cyclophosphamide:Vincristine:Procarbazine:Prednisone was 1:30000:700000:400:8000000:400:6500000:1500000; L428 cells were treated with multiple concentrations for 96 hours, and the killing effect of the combination on L428 was investigated and compared with F0002-ADC; the results showed that the combined BEACOPP scheme had a synergistic effect and achieved better killing effect.

TABLE 14
F0002-ADC combined with BEACOPP in L428 cells

| | | F0002-ADC + BEACOPP | | | |
|---|---|---|---|---|---|
| | | | B | E | A |
| F0002-ADC | | F0002-ADC | (Bleomycin) | (Etoposide) | (Doxorubicin) |
| Concentration | Cell survival | Concentration | Concentration | Concentration | Concentration |
| (nM) | rate (%) | (nM) | (μM) | (μM) | (nM) |
| 0.33 | 29 | 0.041 | 1.238 | 28.88 | 16.50 |
| 0.17 | 33 | 0.021 | 0.638 | 14.88 | 8.50 |
| 0.08 | 35 | 0.010 | 0.300 | 7.00 | 4.00 |
| 0.04 | 46 | 0.005 | 0.150 | 3.50 | 2.00 |
| 0.03 | 65 | 0.004 | 0.112 | 2.62 | 1.50 |
| 0.02 | 79 | 0.002 | 0.075 | 1.75 | 1.00 |
| 0.01 | 87 | 0.001 | 0.038 | 0.88 | 0.50 |
| 0.003 | 88 | 0.0004 | 0.011 | 0.26 | 0.15 |
| | F0002-ADC combined with BEACOPP in L428 cells | | | | |
| | F0002-ADC + BEACOPP | | | | |
| | C | O | P | P | |
| | (Cyclophosphamide) | (Vincristine) | (Procarbazine) | (Prednisone) | |
| | Concentration | Concentration | Concentration | Concentration | Cell survival |
| | (μM) | (nM) | (μM) | (μM) | rate (%) |
| | 330 | 16.50 | 268.12 | 61.88 | 13 |
| | 170 | 8.50 | 138.12 | 31.88 | 22 |
| | 80 | 4.00 | 65 | 15 | 22 |
| | 40 | 2.00 | 32.5 | 7.5 | 33 |
| | 30 | 1.50 | 24.38 | 5.62 | 50 |
| | 20 | 1.00 | 16.25 | 3.75 | 84 |
| | 10 | 0.50 | 8.125 | 1.88 | 90 |
| | 3 | 0.15 | 2.44 | 0.56 | 93 |

(2) In the combination scheme of F0002-ADC+CHOP (Cyclophosphamide, Doxorubicin, Vincristine, Prednisone), the dosing molar ratio of F0002-ADC:Cyclophosphamide:Doxorubicin:Vincristine:Prednisone was 1:1700000:800:11:1400000; L540 cells were treated with multiple concentrations for 96 hours, and the killing effect of the combination on L540 was investigated and compared with F0002-ADC; the results showed that the combined CHOP scheme had a synergistic effect and achieved better killing effect.

TABLE 15
F0002-ADC combined with CHOP in L540 cells

| | | F0002-ADC + CHOP | | | | | |
|---|---|---|---|---|---|---|---|
| | | | C | H | O | P | |
| F0002-ADC | | F0002-ADC | (Cyclophosphamide) | (Doxorubicin) | (Vincristine ) | (Prednisone) | |
| Concentration | Cell survival | Concentration | Concentration | Concentration | Concentration | Concentration | Cell survival |
| (nM) | rate (%) | (nM) | (μM) | (nM) | (nM) | (μM) | rate (%) |
| 0.33 | 27 | 0.066 | 1122 | 52.8 | 0.73 | 92.40 | 14 |
| 0.17 | 43 | 0.034 | 578 | 27.2 | 0.37 | 47.60 | 19 |
| 0.08 | 58 | 0.016 | 272 | 12.8 | 0.18 | 22.40 | 27 |
| 0.04 | 66 | 0.008 | 136 | 6.4 | 0.09 | 11.20 | 45 |
| 0.03 | 69 | 0.006 | 102 | 4.8 | 0.07 | 8.40 | 64 |
| 0.02 | 77 | 0.004 | 68 | 3.2 | 0.04 | 5.60 | 82 |
| 0.01 | 84 | 0.002 | 34 | 1.6 | 0.02 | 2.80 | 83 |
| 0.003 | 97 | 0.001 | 10.2 | 0.48 | 0.01 | 0.84 | 86 |

In the combination scheme of F0002-ADC+CHOP (Cyclophosphamide, Doxorubicin, Vincristine, Prednisone), the dosing molar ratio of F0002-ADC:Cyclophosphamide:Doxorubicin:Vincristine:Prednisone was 1:12000000:1200:600:5500000; Karpas 299 cells were treated with multiple concentrations for 96 hours, and the killing effect of the combination on Karpas 299 was investigated and compared with F0002-ADC; the results showed that the combined CHOP scheme had a synergistic effect and achieved better killing effect.

TABLE 16
F0002-ADC combined with CHOP in Karpas 299 cells

| | | F0002-ADC + CHOP | | | | | |
|---|---|---|---|---|---|---|---|
| | | | C | H | O | P | |
| F0002-ADC | | F0002-ADC | (Cyclophosphamide) | (Doxorubicin) | (Vincristine) | (Prednisone) | |
| Concentration | Cell survival | Concentration | Concentration | Concentration | Concentration | Concentration | Cell survival |
| (nM) | rate (%) | (nM) | (μM) | (nM) | (nM) | (μM) | rate (%) |
| 0.33 | 24 | 0.066 | 792 | 79.20 | 39.60 | 363 | 11 |
| 0.17 | 27 | 0.034 | 408 | 40.80 | 20.40 | 187 | 17 |
| 0.08 | 34 | 0.016 | 192 | 19.20 | 9.60 | 88 | 16 |
| 0.04 | 44 | 0.008 | 96 | 9.60 | 4.80 | 44 | 30 |
| 0.03 | 53 | 0.006 | 72 | 7.20 | 3.60 | 33 | 43 |
| 0.02 | 66 | 0.004 | 48 | 4.80 | 2.40 | 22 | 56 |
| 0.01 | 89 | 0.002 | 24 | 2.40 | 1.20 | 11 | 92 |
| 0.003 | 97 | 0.0006 | 7.20 | 0.72 | 0.36 | 3.30 | 97 |
| 0.001 | 94 | 0.0002 | 2.40 | 0.24 | 0.12 | 1.10 | 98 |

(3) In the combined scheme of F0002-ADC+CVP (Cyclophosphamide, Vinblastine and Prednisone), the molar ratio of F0002-ADC:Cyclophosphamide:Vinblastine:Prednisone was 1:800000:400:1500000; L428 cells were treated with multiple concentrations for 96 hours, the killing results of the combination combined on L428 were examined and compared with F0002-ADC; and the results showed that the combined CVP scheme had better killing effect on L428.

TABLE 17
F0002-ADC combined with CVP in L428 cells

| | | F0002-ADC + CVP | | | | |
|---|---|---|---|---|---|---|
| | | | C | V | P | |
| F0002-ADC | | F0002-ADC | (Cyclophosphamide) | (Vincristine) | (Prednisone) | |
| Concentration | Cell survival | Concentration | Concentration | Concentration | Concentration | Cell survival |
| (nM) | rate (%) | (nM) | (μM) | (nM) | (μM) | rate (%) |
| 0.33 | 29 | 0.082 | 660 | 33 | 124 | 14 |
| 0.17 | 33 | 0.042 | 340 | 17 | 64 | 16 |
| 0.08 | 35 | 0.020 | 160 | 8 | 30 | 20 |
| 0.04 | 46 | 0.010 | 80 | 4 | 15 | 37 |
| 0.03 | 65 | 0.008 | 60 | 3 | 11 | 50 |
| 0.02 | 79 | 0.005 | 40 | 2 | 8 | 73 |
| 0.01 | 87 | 0.002 | 20 | 1 | 4 | 77 |
| 0.003 | 88 | 0.0008 | 6 | 0.30 | 1 | 87 |

In the combined scheme of F0002-ADC+CVP (Cyclophosphamide, Vinblastine and Prednisone), the molar ratio of F0002-ADC:Cyclophosphamide:Vinblastine:Prednisone was 1:12000000:600:5500000; Karpas 299 cells were treated with multiple concentrations for 96 hours, the killing results of the combination on Karpas 299 were examined and compared with F0002-ADC; and the results showed that the combined CVP scheme had a better killing effect on Karpas 299.

TABLE 18
F0002-ADC combined with CVP in Karpas 299 cells

| | | F0002-ADC + CVP | | | | |
|---|---|---|---|---|---|---|
| | | | C | V | P | |
| F0002-ADC | | F0002-ADC | (Cyclophosphamide) | (Vincristine) | (Prednisone) | |
| Concentration | Cell survival | Concentration | Concentration | Concentration | Concentration | Cell survival |
| (nM) | rate (%) | (nM) | (μM) | (nM) | (μM) | rate (%) |
| 0.33 | 24 | 0.082 | 990 | 49.50 | 453.8 | 14 |
| 0.17 | 27 | 0.042 | 510 | 25.50 | 233.8 | 19 |
| 0.08 | 34 | 0.020 | 240 | 12 | 110 | 24 |
| 0.04 | 44 | 0.010 | 120 | 6 | 55 | 28 |
| 0.03 | 53 | 0.008 | 90 | 1.50 | 41.25 | 40 |
| 0.02 | 66 | 0.005 | 60 | 3 | 27.50 | 51 |
| 0.01 | 89 | 0.002 | 30 | 1.50 | 13.75 | 92 |
| 0.003 | 97 | 0.0008 | 9 | 0.45 | 4.13 | 97 |
| 0.001 | 98 | 0.0002 | 3 | 0.15 | 1.38 | 98 |

### Effect Embodiment 8 Acute Toxicity Study

The tolerance difference between F0002-ADC and ADCERIS was compared in the acute toxicity test of monkeys: F0002-ADC 30 mg/kg and ADCERIS 6 mg/kg groups were set, 4 cynomolgus monkeys in each group, half male and half female, were administered intravenously once, and were dissected 4 weeks after recovery. The detection indexes included clinical observation, body weight, food intake, clinical pathology (hematology, serum biochemistry and blood coagulation) and gross anatomy.

The mortality of 6 mg/kg ADCETRIS group was 2/4 on D14 after single administration to cynomolgus monkeys. Histopathological examination showed that the death was caused by drug toxicity, which was related to liver, thymus (decreased immune function) and secondary lung lesions, besides skin and mucous membrane lesions were also observed. No animal died in the F0002-ADC 30 mg/kg group. Clinical observation showed skin erythema after administration in all animals of the two groups, and skin papules (3/4) were also observed on D13 in the Adcetris 6 mg/kg group. The body weight of F0002-ADC 30 mg/kg group decreased significantly (3/4), which was still lower than that before administration after 4 weeks of drug withdrawal, which was consistent with the loss of appetite; the appetite and body weight of ADCETRIS 6 mg/kg group also decreased obviously, but the appetite and body weight of the surviving animals (2/4) returned to normal from D18. The incidence of hematological changes in F0002-ADC 30 mg/kg group was low, mainly a decrease in #NEUT (1 case each on D8, D21 and D28) and a decrease in PLT (1 case each on D5 and D8), which recovered after 7 days. Both of them had reversible changes in skin, hematology and liver function. All lesions can be recovered after 4 weeks after the withdrawal of drug. Cynomolgus monkeys were given a single dose of F0002-ADC 30 mg/kg and ADCETRIS 6 mg/kg; the tolerance of animals to F0002-ADC 30 mg/kg was higher than that of ADCETRIS 6 mg/kg; the tolerable dose of F0002-ADC was 30 mg/kg, and the lethal dose of Adcetris was 6 mg/kg. Compared with ADCETRIS, F0002-ADC has obvious safety advantages, which supports the safety improvement of this drug.

### Effect Embodiment 9 Study on Long Toxicity in Rats

Repeated administration toxicity tests for rats were performed in F0002-ADC blank formulation group, F0002-ADC 5, 10 and 20 mg/kg groups (calculated by DM1, they were 510, 1020 and 2040 μg DM1/m2 respectively), and DM1 0.1 mg/kg group (600 μg DM1/m2); 30 SD rats, half male and half female in each group. There were 4 males and 4 females in each group. The drug was administered by tail vein injection, once on D1, D8, D15 and D22, for 4 times in total. Anatomy was performed at the end of administration and recovery period on D26 and D54 respectively; the evaluation indexes included clinical observation, body weight, food intake, ophthalmic examination, hematology, serum biochemistry, serum electrolyte, blood coagulation and urine analysis, gross anatomy, organ weight, histopathological examination and bone marrow smear examination. F0002-ADC was administered intravenously to SD rats in 4 repeated cycles at a maximum tolerated dose (MTD) of 20 mg/kg; in this test, the toxicity of F0002-ADC was mainly manifested as toxic reactions related to immune hematopoietic organs, liver, kidney and reproductive system. 4 weeks after the withdrawal of drug, the changes of male reproductive system were recovered in the 5 mg/kg group.

### Effect Embodiment 10 Study on Long Toxicity in Monkey

Repeated administration toxicity tests for cynomolgus monkeys were performed with F0002-ADC blank formulation, F0002-ADC 3, 10 and 20 mg/kg, and F0002 monoclonal antibody 20 mg/kg. Fifty cynomolgus monkeys were divided into 5 groups, with 10 in each group, half male and half female. Intravenous infusion once every 21 days was set as one cycle, 4 cycles of continuous administration was performed, the administration rate was 1.5 mL/min, and the recovery period was 6 weeks. The detection indexes include clinical observation, body weight, body temperature, eye examination, food intake, clinical pathology, electrocardiogram, immunogenicity (anti-drug antibody and neutralizing antibody), toxicokinetics, safe pharmacology, local stimulation, lymphocyte typing, circulating immune complex, gross anatomy, histopathological examination and bone marrow smear examination. Cynomolgus monkeys were given F0002-ADC for 4 consecutive cycles, no apparent toxic reaction dose (NOAEL) was 3 mg/kg, the highest non severely toxic dose (HNSTD) was 10 mg/kg; and the minimum lethal dose (MLD) was 20 mg/kg. The main toxic reactions observed with F0002-ADC were skin changes, decreased body weight and food intake, hematological changes (decreased WBC, #NEUT, red lineage and platelets), serum biochemical changes (increased AST, ALP, CK and GLB, decreased ALB and A/G), hemagglutination changes (prolonged APTT and TT, increased FIB), and toxic target organs were sciatic nerve, spinal cord, liver, spleen, kidney, thymus, adrenal gland, breast, sternum (including bone marrow) and seminal vesicles. No significant toxic reactions were observed in the F0002 monoclonal antibody 20 mg/kg group.

The MTD of F0002-ADC was 30 mg/kg in the acute monkey toxicity test, and the unrestricted toxicity dose (HNSTD) was 10 mg/kg in monkey long-term toxicity test. An enzymatically non-degradable linker was used in F0002-ADC; and F0002-ADC is stable in vivo, has low levels of toxic small molecule shedding, and has a low non-specific cytotoxic activity of the active metabolite Lys-MCC-DM1, thus showing a good safety in non-clinical animal studies, and is expected to be a safer and more effective option for targeting CD30 for the treatment of HL, ALCL and CTCL.

Although the above describes specific embodiments of the present disclosure, it should be understood by those skilled in the art that these are merely illustrative embodiments and that a variety of changes or modifications can be made to these embodiments without departing from the principles and substance of the present disclosure. Therefore, the scope of protection of the present disclosure is defined by the appended claims.

1. A method for the treatment of CD30-positive tumors in a subject in need thereof; comprising: administering an effective amount of an antibody conjugate to the subject, wherein, the antibody conjugate is F0002-ADC with a structural general formula of Ab-Lₘ-Yₙ: the CD30-positive tumor is a CD30-positive tumor expressing multidrug resistance gene 1;
wherein, Ab is an anti-human CD30 antibody cAC10, an active fragment thereof, or a variant thereof;
the Ab is only connected with the L;
Y is Mertansine as shown in formula DM1; 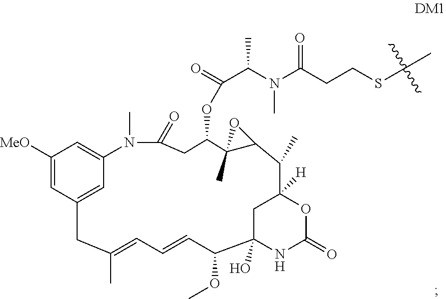
the Y is only connected with the L;
m is 3.3-10; n is 3.3-3.9; and m≥n;
when both ends of L are respectively connected with the Ab and the Y, the L is 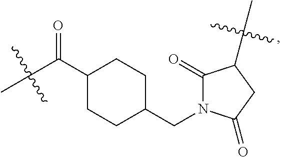 [SMILES: *C(=O)C1CCC(CN2C(=O)CC(*)C2=O)CC1]
its left end forms an amide bond with the amino in lysine of the Ab, and its right end forms a thioether bond with S in the DM1;
when the L is only connected with the Ab, the L is 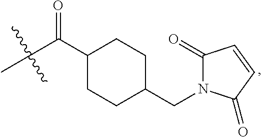 [SMILES: *C(=O)C1CCC(CN2C(=O)C=CC2=O)CC1]
and its left end forms an amide bond with the amino in lysine of the Ab.

2. The method according to claim 1, wherein, the n of the antibody conjugate is 3.6;
or, the similarity between the variant of the anti-human CD30 antibody cAC10 and the amino acid sequence of cAC10 is not less than 90%, and the mutations related to lysine is not more than 80%;
or, the m is equal to the n, the general structural formula is Ab-(L-Y)n, and the structure is as follows: 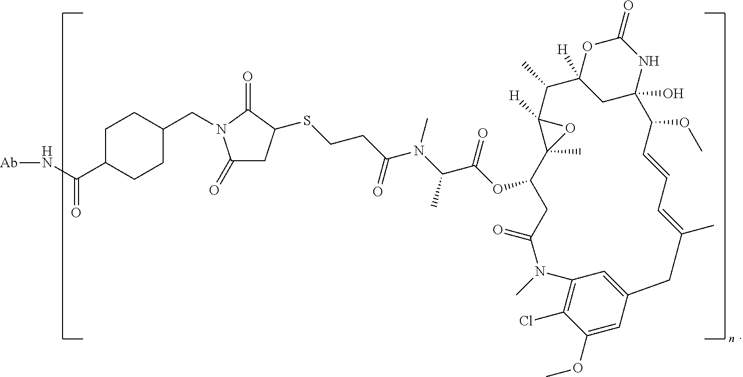
or, the CD30-positive tumor expressing the multidrug resistance gene 1 is Hodgkin lymphoma expressing the multidrug resistance gene 1.

3. The method according to claim 2, wherein, in the F0002-ADC, the m is equal to the n, the general structural formula is Ab-(L-Y)n, and the structure is as follows: is the following structure: 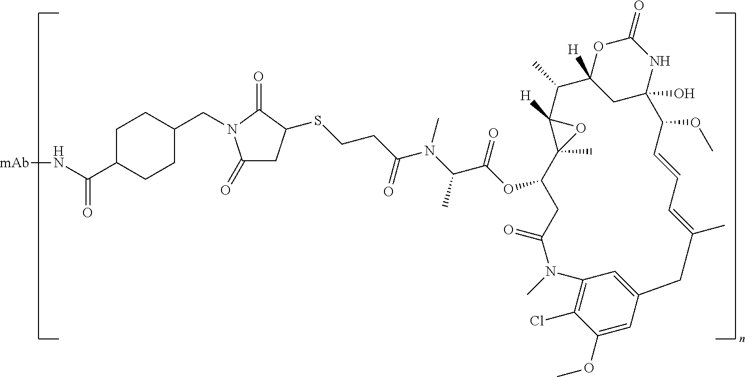
the distribution of different DAR values is as follows:

| D0 | D1 | D2 | D3 | D4 | D5 | D6 | D7 |
|---|---|---|---|---|---|---|---|
| 3% | 10% | 17% | 20% | 18% | 16% | 9% | 7% |

n=3.6;
or, the cells of the Hodgkin lymphoma expressing multidrug resistance gene 1 are CD30-positive Hodgkin lymphoma cells L428 expressing multidrug resistance gene 1 or CD30-positive Hodgkin lymphoma cells L540 expressing multidrug resistance gene 1.

4. A method for the treatment of CD30-positive tumors in a subject in need thereof; comprising: administering an effective amount of an antibody conjugate to the subject, wherein, the antibody conjugate is the F0002-ADC as defined in claim 1; the CD30-positive tumor is a CD30-positive tumor resistant to Adcetris.

5. The method according to claim 4, wherein, the CD30-positive tumor resistant to Adcetris is CD30-positive Hodgkin lymphoma resistant to Adcetris.

6. A method for the treatment of CD30-positive tumors in a subject in need thereof; comprising: administering an effective amount of an antibody conjugate to the subject, wherein, the antibody conjugate is the F0002-ADC as defined in claim 1; the CD30-positive tumor is CD30-positive Hodgkin lymphoma.

7. The method according to claim 6, wherein, the cells of CD30-positive Hodgkin lymphoma are CD30-positive Hodgkin lymphoma cells L428 or CD30-positive Hodgkin lymphoma cells L540.

8. (canceled)

9. A pharmaceutical combination, wherein, the pharmaceutical combination comprises an antibody conjugate X and a substance Y;
the antibody conjugate X is the F0002-ADC as defined in claim 1;
the substance Y is one or more of substances Y1, Y2, Y3, Y4, Y5, Y6, Y7 and Y8;
the substance Y1 is Y1-1, Y1-2 or Y1-3; Y1-1 is Doxorubicin, a pharmaceutically acceptable salt thereof, a solvate thereof, or, a solvate of the pharmaceutically acceptable salt thereof, Y1-2 is Epirubicin, a pharmaceutically acceptable salt thereof, a solvate thereof, or, a solvate of the pharmaceutically acceptable salt thereof, Y1-3 is Daunorubicin, a pharmaceutically acceptable salt thereof, a solvate thereof, or, a solvate of the pharmaceutically acceptable salt thereof;
the substance Y2 is Y2-1, Y2-2, Y2-3, Y2-4 or Y2-5; Y2-1 is Bleomycin, a pharmaceutically acceptable salt thereof, a solvate thereof, or, a solvate of the pharmaceutically acceptable salt thereof, Y2-2 is Boanmycin, a pharmaceutically acceptable salt thereof, a solvate thereof, or, a solvate of the pharmaceutically acceptable salt thereof; Y2-3 is Boningmycin, a pharmaceutically acceptable salt thereof, a solvate thereof, or, a solvate of the pharmaceutically acceptable salt thereof; Y2-4 is Pingyangmycin, a pharmaceutically acceptable salt thereof, a solvate thereof, or, a solvate of the pharmaceutically acceptable salt thereof; Y2-5 is Peplomycin, a pharmaceutically acceptable salt thereof, a solvate thereof, or, a solvate of the pharmaceutically acceptable salt thereof;
the substance Y3 is Y3-1, Y3-2, Y3-3 or Y3-4; Y3-1 is Vinblastine, a pharmaceutically acceptable salt thereof, a solvate thereof, or, a solvate of the pharmaceutically acceptable salt thereof, Y3-2 is Vincristine, a pharmaceutically acceptable salt thereof, a solvate thereof, or, a solvate of the pharmaceutically acceptable salt thereof; Y3-3 is Vinorelbine, a pharmaceutically acceptable salt thereof, a solvate thereof, or, a solvate of the pharmaceutically acceptable salt thereof, Y3-4 is Vindesine, a pharmaceutically acceptable salt thereof, a solvate thereof, or, a solvate of the pharmaceutically acceptable salt thereof;
the substance Y4 is Y4-1 or Y4-2; Y4-1 is Dacarbazine, a pharmaceutically acceptable salt thereof, a solvate thereof, or, a solvate of the pharmaceutically acceptable salt thereof, Y4-2 is Temozolomide, a pharmaceutically acceptable salt thereof, a solvate thereof, or, a solvate of the pharmaceutically acceptable salt thereof;
the substance Y5 is Y5-1 or Y5-2; Y5-1 is Etoposide, a pharmaceutically acceptable salt thereof, a solvate thereof, or, a solvate of the pharmaceutically acceptable salt thereof; Y5-2 is Teniposide, a pharmaceutically acceptable salt thereof, a solvate thereof, or, a solvate of the pharmaceutically acceptable salt thereof;
the substance Y6 is Y6-1 or Y6-2; Y6-1 is Cyclophosphamide, a pharmaceutically acceptable salt thereof, a solvate thereof, or, a solvate of the pharmaceutically acceptable salt thereof, Y6-2 is Ifosfamide, a pharmaceutically acceptable salt thereof, a solvate thereof, or, a solvate of the pharmaceutically acceptable salt thereof;
the substance Y7 is Procarbazine, a pharmaceutically acceptable salt thereof, a solvate thereof, or, a solvate of the pharmaceutically acceptable salt thereof;
the substance Y8 is Y8-1 or Y8-2; Y8-1 is Prednisone, a pharmaceutically acceptable salt thereof, a solvate thereof, or, a solvate of the pharmaceutically acceptable salt thereof; Y8-2 is Prednisone, a pharmaceutically acceptable salt thereof, a solvate thereof, or, a solvate of the pharmaceutically acceptable salt thereof.

10. The pharmaceutical combination according to claim 9, wherein, the substance Y is scheme 1, scheme 2, scheme 3, scheme 4 or scheme 5;
scheme 1 is substances Y1, Y3 and Y4; or,
scheme 2 is substances Y1, Y2, Y3 and Y4; or,
scheme 3 is substances Y2, Y5, Y1, Y6, Y3, Y7 and Y8; or,
scheme 4 is substances Y6, Y1, Y3 and Y8; or,
scheme 5 is substances Y6, Y3 and Y8; or,
or, the antibody conjugate X and “all or part of the substance Y” are administered simultaneously or separately;
or, the pharmaceutical combination is in the form of a mixture of all components, or in the form that each component is independent, or in the form that each component is divided into several groups.

11. A pharmaceutical composition A, wherein, the pharmaceutical composition A comprises the F0002-ADC as defined in claim 1 and a pharmaceutical excipient.

12. A pharmaceutical composition B, wherein, the pharmaceutical composition B comprises the pharmaceutical combination as defined in claim 9 and a pharmaceutical excipient.

13. A method for the treatment of CD30-positive tumors in a subject in need thereof; comprising: administering an effective amount of an antibody conjugate to the subject, wherein, in the method, the antibody conjugate is used in combination with the substance Y; the antibody conjugate is the F0002-ADC as defined in claim 1;
the substance Y is one or more of substances Y1, Y2, Y3, Y4, Y5, Y6, Y7 and Y8;
the substance Y1 is Y1-1, Y1-2 or Y1-3:Y1-1 is Doxorubicin, a pharmaceutically acceptable salt thereof, a solvate thereof, or, a solvate of the pharmaceutically acceptable salt thereof; Y1-2 is Epirubicin, a pharmaceutically acceptable salt thereof, a solvate thereof, or, a solvate of the pharmaceutically acceptable salt thereof; Y1-3 is Daunorubicin, a pharmaceutically acceptable salt thereof, a solvate thereof, or, a solvate of the pharmaceutically acceptable salt thereof;
the substance Y2 is Y2-1, Y2-2, Y2-3, Y2-4 or Y2-5:Y2-1 is Bleomycin, a pharmaceutically acceptable salt thereof, a solvate thereof, or, a solvate of the pharmaceutically acceptable salt thereof; Y2-2 is Boanmycin, a pharmaceutically acceptable salt thereof, a solvate thereof, or, a solvate of the pharmaceutically acceptable salt thereof; Y2-3 is Boningmycin, a pharmaceutically acceptable salt thereof, a solvate thereof, or, a solvate of the pharmaceutically acceptable salt thereof; Y2-4 is Pingyangmycin, a pharmaceutically acceptable salt thereof, a solvate thereof, or, a solvate of the pharmaceutically acceptable salt thereof; Y2-5 is Peplomycin, a pharmaceutically acceptable salt thereof, a solvate thereof, or, a solvate of the pharmaceutically acceptable salt thereof;
the substance Y3 is Y3-1, Y3-2, Y3-3 or Y3-4:Y3-1 is Vinblastine, a pharmaceutically acceptable salt thereof, a solvate thereof, or, a solvate of the pharmaceutically acceptable salt thereof; Y3-2 is Vincristine, a pharmaceutically acceptable salt thereof, a solvate thereof, or, a solvate of the pharmaceutically acceptable salt thereof; Y3-3 is Vinorelbine, a pharmaceutically acceptable salt thereof, a solvate thereof, or, a solvate of the pharmaceutically acceptable salt thereof; Y3-4 is Vindesine, a pharmaceutically acceptable salt thereof, a solvate thereof, or, a solvate of the pharmaceutically acceptable salt thereof;
the substance Y4 is Y4-1 or Y4-2:Y4-1 is Dacarbazine, a pharmaceutically acceptable salt thereof, a solvate thereof, or, a solvate of the pharmaceutically acceptable salt thereof; Y4-2 is Temozolomide, a pharmaceutically acceptable salt thereof, a solvate thereof, or, a solvate of the pharmaceutically acceptable salt thereof;
the substance Y5 is Y5-1 or Y5-2:Y5-1 is Etoposide, a pharmaceutically acceptable salt thereof, a solvate thereof, or, a solvate of the pharmaceutically acceptable salt thereof; Y5-2 is Teniposide, a pharmaceutically acceptable salt thereof, a solvate thereof, or, a solvate of the pharmaceutically acceptable salt thereof;
the substance Y6 is Y6-1 or Y6-2:Y6-1 is Cyclophosphamide, a pharmaceutically acceptable salt thereof, a solvate thereof, or, a solvate of the pharmaceutically acceptable salt thereof; Y6-2 is Ifosfamide, a pharmaceutically acceptable salt thereof, a solvate thereof, or, a solvate of the pharmaceutically acceptable salt thereof;
the substance Y7 is Procarbazine, a pharmaceutically acceptable salt thereof, a solvate thereof, or, a solvate of the pharmaceutically acceptable salt thereof;
the substance Y8 is Y8-1 or Y8-2:Y8-1 is Prednisone, a pharmaceutically acceptable salt thereof, a solvate thereof, or, a solvate of the pharmaceutically acceptable salt thereof; Y8-2 is Prednisone, a pharmaceutically acceptable salt thereof, a solvate thereof, or, a solvate of the pharmaceutically acceptable salt thereof.

14. The method according to claim 13, wherein, the CD30-positive tumor is a CD30-positive lymphoma;
or, the CD30-positive tumor is a CD30-positive tumor expressing multidrug resistance gene 1;
or, the CD30-positive tumor is CD30-positive tumor resistant to Adcetris.

15. A method for the treatment of CD30-positive tumors by administering an effective dose of the pharmaceutical composition as defined in claim 11 to a patient.

16. The method according to claim 15, wherein, the CD30-positive tumor is a CD30-positive lymphoma:
or, the CD30-positive tumor is a CD30-positive tumor expressing multidrug resistance gene 1;
or, the CD30-positive tumor is CD30-positive tumor resistant to Adcetris.

17. The method according to claim 2, wherein, the distribution of different DAR values is as follows:

| D0 | D1 | D2 | D3 | D4 | D5 | D6 | D7 |
|---|---|---|---|---|---|---|---|
| 3% | 10% | 17% | 20% | 18% | 16% | 9% | 7% |

18. The method according to claim 5, wherein, the cells of the CD30-positive tumor resistant to Adcetris are CD30-positive Hodgkin lymphoma cells L428 resistant to Adcetris or CD30-positive Hodgkin lymphoma cells L540 resistant to Adcetris.

19. The pharmaceutical combination according to claim 10, wherein, scheme 1 is substances Y1-1, Y3-2 and Y4-1; or,
scheme 2 is substances Y1-1, Y2-1, Y3-2 and Y4-1; or,
scheme 3 is substances Y2-1, Y5-1, Y1-1, Y6-1, Y3-2, Y7-1 and Y8-1; or,
scheme 4 is substances Y6-1, Y1-1, Y3-2 and Y8-1; or,
scheme 5 is substances Y6-1, Y3-1 and Y8-1.

20. The pharmaceutical combination according to claim 19, wherein, scheme 1 is substances Y1-1, Y3-2 and Y4-1; the molar ratio of the antibody conjugates X:Y1-1:Y3-2:Y4-1 is 1:(400-800):(11-400):(550000-3000000); or,
scheme 2 is substances Y1-1, Y2-1, Y3-2 and Y4-1; the molar ratio of the antibody conjugates X:Y1-1:Y2-1:Y3-2:Y4-1 is 1:(400-800):(30000-45000):(11-400):(550000-3000000); or,
scheme 3 is substances Y2-1, Y5-1, Y1-1, Y6-1, Y3-2, Y7-1 and Y8-1; the molar ratio of the antibody conjugates X:Bleomycin:Etoposide:Doxorubicin:Cyclophosphamide:Vincristine:Procarbazine:Prednisone is 1:30000:700000:400:8000000:400:6500000:1500000; or,
scheme 4 is substances Y6-1, Y1-1, Y3-2 and Y8-1; more preferably the molar ratio of the antibody conjugates X:Y6-1:Y1-1:Y3-2:Y8-1 is 1:(1700000-12000000):(800-1200):(11-600):(550000-1400000); or,
scheme 5 is substances Y6-1, Y3-1 and Y8-1; the molar ratio of the antibody conjugates X:Y6-1:Y3-1:Y8-1 is 1:(8000000-12000000):(400-600):(1500000-5500000).

21. The method according to claim 14, wherein, the CD30-positive lymphoma is CD30-positive Hodgkin lymphoma, CD30-positive anaplastic large cell lymphoma, CD30-positive diffuse histiocytic lymphoma or CD30-positive cutaneous T cell lymphoma;
or, the CD30-positive tumor is a CD30-positive Hodgkin lymphoma expressing multidrug resistance gene 1;
or, the CD30-positive tumor is CD30-positive Hodgkin lymphoma resistant to Adcetris.
